(19) United States
(12) Patent Application Publication
(10) Pub. No.: US 2005/0124071 A1
(43) Pub. Date: Jun. 9, 2005

(54) Methods and compositions for diagnosing musculoskeletal, arthritic and joint disorders by biomarker dating

(76) Inventor: Virginia Byers Kraus, Hillsborough, NC (US)

Correspondence Address: MYERS BIGEL SIBLEY & SAJOVEC, PO BOX 37428, RALEIGH, NC, 27627, US

(21) Appl. No.: 10/952,638

(22) Filed: Sep. 29, 2004

Related U.S. Application Data
(60) Provisional application No. 60/507,599, filed on Sep. 30, 2003.

(51) Int. Cl. G06F 19/00; G01N 33/48; G01N 33/50; G01N 33/20

(52) U.S. Cl. 436/84; 702/19

(57) ABSTRACT

The present invention provides a method of determining, in a sample, the proportion of a total amount of a molecule that is derived from catabolism due to the presence of age-related molecular alterations on the molecule, comprising: a) determining the total amount of the molecule in the sample; b) determining the amount of the molecule in the sample that contains D-aspartate; and c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule. Further provided is a method of diagnosing a musculoskeletal, arthritic or joint disorder in a subject and/or identifying a subject at risk for developing such a disorder, comprising: a) measuring an amount of D-aspartate and/or an advanced glycation end product in a sample of the subject; and b) comparing the amount of D-aspartate and/or advanced glycation end product in the sample of (a) with an amount of D-aspartate and/or advanced glycation end product in a sample of a control subject, whereby an increased amount of D-aspartate and/or advanced glycation end product in the sample of the subject as compared to the amount of D-aspartate and/or advanced glycation end product in the sample of the control subject is diagnostic of a musculoskeletal, arthritic or joint disorder in the subject and/or identifies a subject at risk of developing such a disorder.

### PRIORITY STATEMENT

This application claims the benefit, under 35 U.S.C. § 119(e), of U.S. Provisional Application No. 60/507,599, filed Sep. 30, 2003, which application is herein incorporated by reference in its entirety.

### STATEMENT OF GOVERNMENT SUPPORT

This invention was made with Government support under grant number U01 AR050898-01 from the National Institutes of Health. The Government has certain rights to this invention.

### FIELD OF THE INVENTION

The present invention provides compositions and methods for diagnosing, prognosing, and/or identifying subjects at risk for musculoskeletal, arthritic and/or joint disorders.

### BACKGROUND OF THE INVENTION

Osteoarthritis (OA) is a degenerative joint disease that is one of the oldest and most common types of arthritis. It can be characterized by the breakdown of the cartilage in the joints. There are many factors that can cause osteoarthritis. Although age is a risk factor, research has shown that osteoarthritis is not an inevitable part of aging. Obesity may lead to osteoarthritis of the knees. In addition, people with joint injuries due to sports, work-related activity or accidents may be at increased risk of developing osteoarthritis. Additionally, genetics has a role in the development of osteoarthritis, particularly in the hands. Some people may be born with defective cartilage or with slight defects in the way that joints fit together. As a person ages, these defects may cause early cartilage breakdown in the joint.

Currently, diagnosis of osteoarthritis is based on a physical exam and history of symptoms. Radiographs or X-rays are used to confirm diagnosis, but they detect late-stage and irreversible disease. The present invention overcomes previous shortcomings in the art by providing methods and compositions for earlier detection of disease and identification of individuals at risk for developing progressive OA, thereby facilitating early interventions, primary prevention of the disease, and treatment of preclinical OA when the disease is potentially reversible.

Similar shortcomings in the art also exist for diagnosing and monitoring the stages of the many other forms of arthritis, including rheumatoid arthritis, ankylosing spondylitis and gout to name a few. As for osteoarthritis, the primary clinical means of detecting and monitoring disease is by radiographs. The present invention overcomes existing limitations by providing methods for early detection and monitoring of the joint damage of these arthritides.

### SUMMARY OF THE INVENTION

In general, the present invention provides methods of diagnosing, prognosing, and/or screening for musculoskeletal, arthritic and joint disorders and diseases.

A further aspect of the present invention is a method of diagnosing, prognosing, screening and/or monitoring therapies for osteoarthritis.

Another aspect of the present invention provides a method of measuring the fraction of D-aspartate and/or L-aspartate in joint tissue molecules such as collagen and aggrecan.

A still further aspect of the present invention provides a method of detecting distinct pools of joint tissue molecules and measuring the turnover rates for joint tissue molecules.

A further aspect of the present invention provides a method of determining the degree of catabolism of pools of joint tissue molecules.

A still further aspect of the present invention provides biomarkers for determining subjects afflicted with or at risk for musculoskeletal, arthritic and/or joint disorders, such as osteoarthritis.

An additional aspect of the present invention relates to a method of evaluating catabolic and/or anabolic processes and relating the catabolic and/or anabolic processes to the risk of or affliction with musculoskeletal, arthritic and/or joint disorders, such as osteoarthritis.

A still further aspect of the present invention provides antibodies, such as monoclonal antibodies, which specifically bind D-aspartate and/or L-aspartate.

A further aspect of the present invention relates to an enzyme-linked immunosorbent assay (ELISA)-type assay for diagnoses, prognoses and/or screening of musculoskeletal, arthritic and/or joint disorders, such as osteoarthritis.

A further aspect of the present invention provides a kit for diagnosing, prognosing, and/or screening for musculoskeletal, arthritic and/or joint disorders, such as osteoarthritis.

A still further aspect of the present invention provides a method of diagnosing, prognosing, and/or screening for musculoskeletal, arthritic and/or joint disorders, such as osteoarthritis comprising measuring the fraction of D-aspartate and/or L-aspartate in joint tissue molecules in a biological sample.

Another aspect of the present invention provides use of a means of measuring the fraction of D-aspartate and/or L-aspartate in joint tissue molecules in determining whether a subject is afflicted with or at risk of developing musculoskeletal, arthritic and/or joint disorders, such as osteoarthritis.

Additional embodiments of this invention include a method of determining, in a sample, the proportion of a total amount of a molecule that is derived from catabolism due to the presence of age-related molecular alterations on the molecule, comprising: a) determining the total amount of the molecule in the sample; b) determining the amount of the molecule in the sample that contains D-aspartate and/or an advanced glycation end product; and c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

Furthermore, the present invention provides a method of diagnosing a musculoskeletal, arthritic and/or joint disorder in a subject, comprising; a) measuring an amount of D-aspartate and/or an advanced glycation end product in a sample of the subject; and b) comparing the amount of D-aspartate and/or advanced glycation end product in the sample of (b) with an amount of D-aspartate and/or advanced glycation end product in a sample of a control subject, whereby an increased amount of D-aspartate and/or advanced glycation end product in the sample of the subject as compared to the amount of D-aspartate and/or advanced glycation end product in the sample of the control subject is diagnostic of a musculoskeletal, arthritic and/or joint disorder in the subject.

In addition, the present invention provides a method of diagnosing a musculoskeletal, arthritic and/or joint disorder in a subject, comprising: a) determining the proportion of a total amount of a molecule in the subject that is derived from a catabolic process comprising: i) determining the total amount of the molecule in a joint tissue sample from the subject; ii) determining the amount of the molecule in the sample that contains D-aspartate and/or an advanced glycation end product; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic and/or joint disorder in the subject.

In further embodiments, the present invention provides a method of identifying a subject at risk of developing a musculoskeletal, arthritic and/or joint disorder, comprising: a) measuring an amount of D-aspartate and/or an advanced glycation end product in a sample of the subject; and b) comparing the amount of D-aspartate and or advanced glycation end product in the sample of (b) with an amount of D-aspartate (D-Asp) and/or advanced glycation end product in a sample of a control subject, whereby an increased amount of D-aspartate and/or advanced glycation end product in the sample of the subject as compared to the amount of D-aspartate and/or advanced glycation end product in the sample of the control subject identifies a subject at risk of developing a musculoskeletal, arthritic and/or joint disorder.

Further provided is a method of identifying a subject at risk of developing a musculoskeletal, arthritic and/or joint disorder, comprising: a) determining the proportion of a total amount of a molecule in the subject that is derived from a catabolic process comprising: i) determining the total amount of the molecule in a joint tissue sample from the subject; ii) determining the amount of the molecule in the sample that contains D-aspartate and/or an advanced glycation end product; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject identifies a subject at increased risk of developing a musculoskeletal, arthritic and/or joint disorder.

In addition, the present invention provides a method of identifying a subject with a musculoskeletal, arthritic and/or joint disorder as having a poor prognosis, comprising: a) establishing a correlation between an absolute amount, or a proportion in a total amount of a joint tissue molecule, of D-Asp and/or an advanced glycation end product, from test subjects with a musculoskeletal, arthritic or joint disorder and who have and/or had a poor prognosis; and b) detecting in the subject the absolute amount, or the proportion in a total amount of a joint tissue molecule, of D-Asp and/or an advanced glycation end product correlated with a poor prognosis according to step (a), thereby identifying the subject as having a poor prognosis.

Further provided is a method of monitoring the therapeutic efficacy of a treatment regimen for a musculoskeletal, arthritic and/or joint disorder in a subject, comprising: detecting, in a sample from the subject, the amount of D-Asp and/or an advanced glycation end product, either as an absolute amount in the sample or as a proportion of a total amount of a joint tissue molecule in the sample, over time prior to and/or during the treatment regimen, whereby a decrease in the amount of D-Asp and/or advanced glycation end products after the onset of the treatment regimen and/or over time during the treatment regimen indicates therapeutic efficacy of the treatment regimen.

In additional embodiments, the present invention provides a method of identifying an effective treatment regimen for a musculoskeletal, arthritic and/or joint disorder in a subject, comprising: detecting, in a sample from the subject, the amount of D-Asp and/or an advanced glycation end product, either as an absolute amount in the sample or as a proportion of a total amount of a joint tissue molecule in the sample, prior to and/or over time during the treatment regimen, whereby a decrease in the amount of D-Asp and/or advanced glycation end product after the onset of the treatment regimen and/or over time during the treatment regimen identifies an effective treatment regimen.

### DETAILED DESCRIPTION OF THE INVENTION

The foregoing and other aspects of the present invention will now be described in more detail with respect to other embodiments described herein and as according to the National Institutes of Health grant application for Grant No. U01 AR050898-01, incorporated by reference in its entirety herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention.

As used in the description of the invention and the claims set forth herein, the singular forms “a,” “an,” and “the” are intended to include the plural forms as well, unless the context clearly indicates otherwise. For example, “an advanced glycation end product” can mean a single advanced glycation end product or a plurality of advanced glycation end products.

All publications, patent applications, patents and other references cited herein are incorporated by reference in their entireties for the teachings relevant to the sentence and/or paragraph in which the reference is presented.

In view of the foregoing, embodiments according to the present invention relate to methods of diagnosing, prognosing, and/or screening for musculoskeletal, arthritic and/or joint diseases and other joint derangement, including but not limited to, osteoarthritis (OA), rheumatoid arthritis (RA) and injury.

Further embodiments relate to measuring the fraction of D-aspartate and/or L-aspartate in molecules such as joint tissue molecules, including but not limited to, collagen, such as type II collagen, and aggrecan. Molecules of this invention can be derived from body fluids, including but not limited to serum, plasma, urine, and synovial fluid.

Additional embodiments of the present invention relate to methods of detecting distinct pools of molecules of this invention that contain biomarkers and/or have the potential for containing biomarkers and measuring the turnover rates for such molecules. Embodiments of the present invention further provide a method of determining the degree of catabolism of pools of molecules of this invention. For example, turnover rates can range from 100-400 years for collagen and from 3-25 years for aggrecan. Additionally, the fraction of D-aspartate in the fragments derived from joint tissue molecules present in, for example, serum, plasma, urine and synovial fluid can reflect the degree of catabolism of the oldest pool of joint tissue molecules.

As one example, biomarker dating with D-aspartate allows for the assessment of a state of high turnover of cartilage accompanied by the risk of rapid cartilage matrix loss. Most importantly, D-aspartate measurements allow for the differentiation of a deleterious and pathological state from a high turnover state of cartilage unaccompanied by significant loss of matrix substance.

Embodiments of the present invention provide biomarkers for determining subjects afflicted with or at risk for musculoskeletal, arthritic and/or joint disorders such as osteoarthritis. Additionally, embodiments of the present invention relate to methods of evaluating catabolic and/or anabolic processes and relating the catabolic and/or anabolic processes to the risk or detection of musculoskeletal, arthritic and/or joint disorders such as osteoarthritis.

Embodiments of the present invention further provide antibodies, such as polyclonal and monoclonal antibodies, which specifically recognize and bind D-aspartate and/or L-aspartate (L-Asp). Further embodiments of the present invention relate to immunoassays, such as, for example, enzyme-linked immunosorbent assays (ELISA), immunoprecipitation assays, immunohistochemical assays, enzyme immunoassays (EIA), agglutination assays, precipitation/flocculation assays, immunoblots (Western blot; dot/slot blot, etc.), radioimmunoassays (RIA), immunodiffusion assays, immunofluorescence assays (e.g., FACS); chemiluminescence assays, antibody library screens, expression arrays, etc., for diagnosis of, prognosis of and/or screening for musculoskeletal, arthritic and joint diseases and disorders of this invention.

Embodiments of the present invention further include kits for diagnosing, prognosing, and/or screening for the diseases and disorders of this invention. The kits can contain reagents as described herein for diagnosing, prognosing, and/or screening for the diseases and disorders described herein and printed instructions for use thereof, in a container or package. Such reagents can include, but are not limited to, monoclonal and/or polyclonal antibodies to D-Asp, monoclonal and/or polyclonal antibodies to L-Asp, monoclonal and/or polyclonal antibodies to an advanced glycation end product of this invention (pooled or single), control antigens, calibration antigens, secondary antibodies to detect antigen/antibody complex formation; detection reagents, buffers, diluents, slides, vessels, chambers, mixers, plates, vials, etc., as would be well known to one of ordinary skill in the art to conduct immunoassay protocols.

Subjects suitable to be diagnosed, prognosed, or screened according to the methods of the present invention include, but are not limited to, avian and mammalian subjects, and in many embodiments are mammalian. Mammals of the present invention include, but are not limited to, canines, felines, bovines, caprines, equines, ovines, porcines, rodents (e.g., rats, mice and guinea pigs), lagomorphs, primates, humans, and the like, and mammals in utero. Any mammalian subject in need of being diagnosed, prognosed, or screened according to the present invention is suitable. Human subjects are used in many embodiments. Human subjects of both genders and at any stage of development (i.e., neonate, infant, juvenile, adolescent, and adult) can be diagnosed, prognosed, and/or screened according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the diagnosis, prognosis, and/or screening of human subjects or biological samples therefrom, but the invention can also be carried out on animal subjects or biological samples therefrom, particularly mammalian subjects such as mice, rats, guinea pigs, dogs, cats, livestock and horses for veterinary purposes, and for drug screening and drug development purposes.

In further embodiments, the present invention provides methods and compositions directed to the identification of age related alterations of protein that can be used to identify circulating molecules indicative of extracellular matrix catabolism. Such alterations include, for example, advanced glycation end products as well as racemized amino acids. Glycation, like aspartate racemization, is a time dependent event and cartilage extracellular matrix accumulates these changes. Pentosidine is one advanced glycation end product that can be measured.

Pentosidine, the most extensively studied form of advanced glycation end product, increases in a variety of tissues with age, including the skin, kidney and lens [60]. Cartilage extracellular matrix also accumulates advanced glycation end products over time and thus shows an increase in these molecular changes within cartilage with ageing. Pentosidine is present in serum and synovial fluid [63]. Serum pentosidine has been shown to be elevated above control levels in some, but not all, patients with rheumatoid arthritis (RA), total serum pentosidine was not elevated in all patients with RA. In one study of 60 patients [60], only ˜25% of the RA patients had elevated serum pentosidine levels. The authors stated that it is “unclear why only some patients show increased pentosidinemia,” and that “circulating AGEs [advanced glycation end products] may be irrelevant by-products” in RA. Moreover, tissue and serum levels of pentosidine also increase with diabetes and renal disease [60]. Thus, these two prevalent medical conditions can confound the interpretation of a pentosidine level. Serum pentosidine has been categorized as a marker of glycoxidation in diabetes and as a more general marker of oxidative stress in different pathologies. AGEs in general have been considered measures of oxidative damage [64].

The present invention overcomes previous difficulties in the evaluation of a plasma, serum or urine AGE level for clinically useful purposes. In the methods described herein, molecules specific to joint tissue are measured and the amount of these molecules possessing advanced glycation end products is quantified, as well as the amount of these molecules without advanced glycation end products. Comparing the levels of AGE-containing joint tissue molecules in patients with joint disorders versus age-matched controls, it is possible to focus in on joint catabolic processes within a potential sea of irrelevant AGE containing molecules. Validation can also be carried out, for example, in a population with diabetes (with and without joint disorders) and in a population with renal impairment (with and without joint disorders), to establish the appropriate threshold values representative of pathological joint turnover in these specialized populations. In this way the measurement of advanced glycation end products can be made very specific to detect catabolism of joint tissues, thereby distinguishing an elevated AGE level due to a musculoskeletal, arthritic or joint disorder from an elevated AGE level resulting from some other unrelated physiological effect.

In many cases, a particular molecule associated with joint disorders originates not only from cartilage but from other tissues as well, including tissues from outside the joint. For example, this is the situation with COMP, which is present in cartilage, synovium, tendon, bone and the vascular adventitia. The accumulation of D-Asp and advanced glycation end products in a molecule only occurs in tissues where the residence time is sufficient to produce these age related molecular alterations. Thus, in one aspect, this invention provides a means for distinguishing molecules of cartilage origin, where residence time is the longest, from a pool of similar molecules originating in other tissues where turnover is rapid and residence time by definition is short. Turnover, as used herein, describes the net effect of synthesis and breakdown of a molecule of interest.

Thus, in one aspect, the present invention is based on the concept of “biomarker dating,” which is the identification of proteins in the circulation and in body fluids with non-enzymatic, age-related molecular alterations, in order to distinguish a pool of molecules resulting from tissue catabolism, from a pool resulting from recent synthesis. This allows for distinguishing between the pathological process of joint tissue breakdown from a) a condition of high turnover without net breakdown, and b) a condition of synthesis without incorporation in the extracellular matrix resulting in loss of newly synthesized molecules to the circulation and body fluids. A variety of molecular alterations accumulate in proteins in a non-enzymatic and time-dependent manner. The presence of these age-related molecular alterations represent natural labels of a particular molecule's residence time in a particular tissue. Racemization of aspartate is one such alteration. Glycation is another type of alteration that is age-dependent. Spontaneous chemical reactions between proteins and sugars lead to non-enzymatically formed crosslinks that accumulate in long-lived proteins. These products, termed advanced glycation end products, accumulate in tissues as a function of time and sugar concentration. Cartilage extracellular matrix accumulates these senescent crosslinks over time and thus shows an increase in these molecular changes within cartilage with ageing. Non-enzymatic glycation yields multiple products, including pentosidine, N(epsilon)-(carboxymethyl)lysine, N(epsilon)-(carboxyethyl)lysine, imidazolone, and pyrraline.

Thus, in a particular embodiment, the present invention provides a method of determining, in a sample, the proportion of a total amount of a molecule that is derived from catabolism due to the presence of age-related molecular alterations on the molecule, comprising: a) determining the total amount of the molecule in the sample; b) determining the amount of the molecule in the sample that contains D-aspartate; and c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

In an additional embodiment, the present invention provides a method of determining, in a sample, the proportion of a total amount of a molecule that is derived from catabolism due to the presence of age-related molecular alterations on the molecule, comprising: a) determining the total amount of the molecule in the sample; b) determining the amount of the molecule in the sample that contains an advanced glycation end product; and c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

Further provided herein is a method of determining, in a sample, the proportion of a total amount of a molecule that is derived from catabolism due to the presence of age-related molecular alterations on the molecule, comprising: a) determining the total amount of the molecule in the sample; b) determining the amount of the molecule in the sample that contains D-aspartate and the amount of the molecule in the sample that contains an advanced glycation end product; and c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

In these methods, by determining the proportion of the total amount of a molecule that is derived from catabolism due to the presence of age-related molecular alterations on the molecule, other measurements can be obtained as well. For example, it is also possible to determine, according to these methods, the proportion of old molecules undergoing turnover relative to overall turnover when the amount of the molecule as determined in step (a) is an amount derived from total catabolism or overall turnover. In this circumstance, the amount of the molecule as determined in step (b) is the proportion of old molecules undergoing turnover relative to overall turnover. It is also possible to determine the relative amounts of turnover of young and old molecules when the amount of the molecule as determined in step (a) is an amount derived from overall turnover and the amount as determined in step (b) is the amount of turnover of old molecules. In this circumstance, the difference between the amount of (a) and the amount of (b) is the amount of turnover of young molecules, which can include molecules recently synthesized and then catabolized, as well as molecules recently synthesized that fail to incorporate or integrate appropriately into the tissue, thus representing turnover due to an ineffective anabolic process.

Thus, the present invention further provides a method of determining the proportion of old molecules undergoing turnover relative to overall turnover of a molecule in a sample, comprising: a) determining the total amount of the molecule in the sample, thereby determining the overall turnover rate; b) determining the amount of the molecule in the sample that contains D-aspartate and/or the amount of the molecule in the sample that contains an advanced glycation end product; and c) calculating the proportion of the amount of the molecule of step (b) relative to the total amount of the molecule as determined in step (a), thereby determining the proportion of old molecules undergoing turnover relative to overall turnover of the molecule.

Additionally provided herein is a method of determining the relative amount of turnover of young and old molecules in a total amount of a molecule in a sample, comprising: a) determining the total amount of the molecule in the sample; b) determining the amount of the molecule in the sample that contains D-aspartate and/or the amount of the molecule in the sample that contains an advanced glycation end product, thereby determining the amount of turnover of old molecules; and c) calculating the difference between the amount of step (a) and the amount of step (b), thereby determining the amount of turnover of young molecules.

In yet further embodiments, the present invention provides a method of diagnosing a musculoskeletal, arthritic or joint disorder in a subject, comprising: a) measuring an amount of D-aspartate in a sample of the subject; and b) comparing the amount of D-aspartate in the sample of (a) with an amount of D-aspartate in a sample of a control subject, whereby an increased amount of D-aspartate in the sample of the subject as compared to the amount of D-aspartate in the sample of the control subject is diagnostic of a musculoskeletal, arthritic or joint disorder in the subject.

Also provided herein is a method of diagnosing a musculoskeletal, arthritic or joint disorder in a subject, comprising; a) measuring an amount of an advanced glycation end product in a sample of the subject; and b) comparing the amount of the advanced glycation end product in the sample of (a) with an amount of an advanced glycation end product in a sample of a control subject, whereby an increased amount of an advanced glycation end product in the sample of the subject as compared to the amount of an advanced glycation end product in the sample of the control subject is diagnostic of a musculoskeletal, arthritic or joint disorder in the subject.

Further provided herein is a method of diagnosing a musculoskeletal, arthritic or joint disorder in a subject, comprising: a) measuring an amount of D-aspartate and an amount of an advanced glycation end product in a sample of the subject; and b) comparing the amount of the D-aspartate and the amount of the advanced glycation end product in the sample of (a) with an amount of D-aspartate and an amount of an advanced glycation end product in a sample of a control subject, whereby an increased amount of D-aspartate and of an advanced glycation end product in the sample of the subject as compared to the amount D-aspartate and of an advanced glycation end product in the sample of the control subject is diagnostic of a musculoskeletal, arthritic or joint disorder in the subject.

In additional embodiments, the present invention provides a method of diagnosing a musculoskeletal, arthritic or joint disorder in a subject, comprising: a) determining the proportion of a total amount of a molecule in the subject that is derived from a catabolic process comprising: i) determining the total amount of the molecule in a joint tissue sample from the subject; ii) determining the amount of the molecule in the sample that contains D-aspartate; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic or joint disorder in the subject.

Also provided herein is a method of diagnosing a musculoskeletal, arthritic or joint disorder in a subject, comprising: a) determining the proportion of a total amount of a molecule in the subject that is derived from a catabolic process comprising: i) determining the total amount of the molecule in a joint tissue sample from the subject; ii) determining the amount of the molecule in the sample that contains an advanced glycation end product; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic or joint disorder in the subject.

In addition, the present invention provides a method of diagnosing a musculoskeletal, arthritic or joint disorder in a subject, comprising: a) determining the proportion of a total amount of a molecule in the subject that is derived from a catabolic process comprising: i) determining the total amount of the molecule in a joint tissue sample from the subject; ii) determining the amount of the molecule in the sample that contains D-aspartate and the amount of the molecule in the sample that contains an advanced glycation end product; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic or joint disorder in the subject.

In still further embodiments, the present invention provides a method of identifying a subject at risk of developing a musculoskeletal, arthritic or joint disorder, comprising; a) measuring an amount of D-aspartate in a sample of the subject; and b) comparing the amount of D-aspartate in the sample of (a) with an amount of D-aspartate in a sample of a control subject, whereby an increased amount of D-aspartate in the sample of the subject as compared to the amount of D-aspartate in the sample of the control subject identifies a subject at risk of developing a musculoskeletal, arthritic or joint disorder.

Furthermore, the present invention provides a method of identifying a subject at risk of developing a musculoskeletal, arthritic or joint disorder, comprising; a) measuring an amount of an advanced glycation end product in a sample of the subject; and b) comparing the amount of the advanced glycation end product in the sample of (a) with an amount of an advanced glycation end product in a sample of a control subject, whereby an increased amount of an advanced glycation end product in the sample of the subject as compared to the amount of an advanced glycation end product in the sample of the control subject identifies a subject at risk of developing a musculoskeletal, arthritic or joint disorder.

In addition, the present invention provides a method of identifying a subject at risk of developing a musculoskeletal, arthritic or joint disorder, comprising; a) measuring an amount of D-aspartate and an amount of an advanced glycation end product in a sample of the subject; and b) comparing the amount of the D-aspartate and the amount of the advanced glycation end product in the sample of (a) with an amount of D-aspartate and an amount of an advanced glycation end product in a sample of a control subject, whereby an increased amount of D-aspartate and of an advanced glycation end product in the sample of the subject as compared to the amount D-aspartate and of an advanced glycation end product in the sample of the control subject identifies a subject at risk of developing a musculoskeletal, arthritic or joint disorder.

Also provided herein is a method of identifying a subject at risk of developing a musculoskeletal, arthritic or joint disorder, comprising: a) determining the proportion of a total amount of a molecule in the subject that is derived from a catabolic process comprising: i) determining the total amount of the molecule in a joint tissue sample from the subject; ii) determining the amount of the molecule in the sample that contains D-aspartate; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject identifies a subject at increased risk of developing a musculoskeletal, arthritic or joint disorder.

Furthermore, the present invention provides a method of identifying a subject at risk of developing a musculoskeletal, arthritic or joint disorder, comprising: a) determining the proportion of a total amount of a molecule in the subject that is derived from a catabolic process comprising: i) determining the total amount of the molecule in a joint tissue sample from the subject; ii) determining the amount of the molecule in the sample that contains an advanced glycation end product; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject identifies a subject at risk of developing a musculoskeletal, arthritic or joint disorder.

Additionally provided is a method of identifying a subject at risk of developing a musculoskeletal, arthritic or joint disorder, comprising: a) determining the proportion of a total amount of a molecule in the subject that is derived from a catabolic process comprising: i) determining the total amount of the molecule in a joint tissue sample from the subject; ii) determining the amount of the molecule in the sample that contains D-aspartate and the amount of the molecule in the sample that contains an advanced glycation end product; and iii) calculating the proportion of the amount of the molecule of step (ii) relative to the total amount of the molecule as determined in step (i), thereby determining the proportion derived from a catabolic process; and b) comparing the proportion of the molecule in the subject that is derived from a catabolic process with the proportion of the molecule in a control subject that is derived from a catabolic process, whereby an increased proportion in the subject as compared to the proportion in the control subject is identified as a subject at risk of developing a musculoskeletal, arthritic or joint disorder.

Levels of D-aspartate and/or advanced glycation end products can be measured according to protocols well known in the art and as described in the Examples section herein, such as, for example, HPLC, immunoassay, mass spectrometry and microarray. In the methods of diagnosing and/or identifying a subject at risk as described herein, the amount of D-aspartate and/or advanced glycation end product measured can be an absolute amount or it can be a proportion of a total amount in a sample of a subject of this invention. An increase in an absolute amount or in a proportion of a total amount in a subject as compared to an absolute amount or a proportion of a total amount in a control subject of this invention can be any increase determined according to the methods of this invention to be relevant.

Thus, in some embodiments the increase can be any value that is greater than the value of the control and in other embodiments, the increase can be an increase over a threshold value. For example, in some embodiments, for an increase to be diagnostic and/or predictive of risk, it can be an increase of at least 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250 or 300% over the value of the control. It is further contemplated that in some embodiments, a proportion of a molecule containing D-aspartate and/or an advanced glycation end product in a total amount of the molecule that is diagnostic and/or predictive of risk can be described as a percentage of the total amount of the molecule being measured. Thus, for example, in a sample containing a molecule, a proportion of the total amount of the molecule that contains D-aspartate and/or an advanced glycation end product that is at least 0.5, 1, 2, 3, 4, 5, 6, 7, 8, 9, 10, 15, 20, 25, 30, 35, 40, 45, 50, 55, 60, 65, 70, 75, 80, 85, 90, 95, 98, 99 or 100% of the total amount of the molecule can be diagnostic and/or predictive of risk of developing a disorder and/or disease of this invention.

In some circumstances, as described herein, an absolute value can be diagnostic and/or predictive of risk. In all circumstances, in the methods of this invention, evaluating a proportion of D-aspartate and/or an advanced glycation end product of a total amount of a molecule in a sample from a subject of this invention allows for specific diagnosis and/or prediction of risk because such an evaluation allows for distinguishing molecules that result unequivocally from tissue catabolism from molecules that result from recent synthesis.

In the embodiments of this invention, the sample can be a biological sample from a subject of this invention and can be, for example, a body fluid, such as synovial fluid, serum, plasma, urine, fluid from lavage of a joint, or any other fluid that contains proteins, protein fragments (e.g., collagen breakdown products) and/or molecules having and/or potentially having D-aspartate and/or advanced glycation end products that can be measured in the assays of this invention. The sample can also be a cell, cell fraction, cell lysate, tissue, tissue fragment or tissue homogenate, which can be from a subject of this invention.

As noted above, the sample of this invention can be any sample containing molecules having or potentially having D-aspartate and/or advanced glycation end products that can be measured in the assays of this invention. In some embodiments, such molecules can be, but are not limited to, joint tissue fragments, joint tissue proteins, joint tissue fragments and joint tissue molecules, which can be, for example, cartilage oligomeric matrix protein (COMP), link protein, type I, II, III, V, VI, IX, X, XI and XII collagens, aggrecan, glycosaminoglycan, link protein and any combination thereof, as well as any other joint tissue molecule or joint tissue fragment now known or later identified.

A variety of advanced glycation end products can be measured in the methods of this invention, including, but not limited to, pentosidine, N(epsilon)-(carboxymethyl)lysine, N(epsilon)-(carboxyethyl)lysine, imidazolone, and pyrraline and any combination thereof, as well as any advanced glycation end products now known or later identified that can be measured in the assays of this invention.

The musculoskeletal, arthritic or joint disease or disorder of this invention includes, but is not limited to, osteoarthritis, rheumatoid arthritis, psoriatic arthritis, ankylosing spondylitis, gout, crystalline arthritis, any arthritis of unknown etiology, joint injury, relapsing polychondritis and any combination thereof, as well as any such disorder or disease now known or later identified that is associated with joint tissue molecules that may have an increase in the total amount or in a proportional amount of D-aspartate and/or one or more advanced glycation end products.

In further embodiments, the present invention provides a method of identifying a subject with a musculoskeletal, arthritic or joint disorder as having a poor prognosis, comprising: a) establishing a correlation between an absolute amount, or a proportion in a total amount of a joint tissue molecule, of D-Asp and/or an advanced glycation end product, from test subjects with a musculoskeletal, arthritic and/or joint disorder and who have or had a poor prognosis; and b) detecting in the subject the absolute amount, or the proportion in a total amount of a joint tissue molecule, of D-Asp and/or an advanced glycation end product correlated with a poor prognosis according to step (a), thereby identifying the subject as having a poor prognosis. For example, a correlation can be made between an absolute or proportional amount of D-Asp and/or advanced glycation end products and a degree and/or amount of degeneration, deterioration, deformity, atrophy, loss of function, structural deterioration represented by radiograph, MRI techniques, bone scan and ultrasound, joint crepitus, pain and joint symptoms, etc., as would be indicative of a poor prognosis. A correlation would be made by evaluating a sufficient number of test subjects to allow for a statistical analysis of the association of a particular amount of D-aspartate and/or advanced glycation end products and clinical parameters associated with a poor prognosis as would be recognized by one skilled in the art. Protocols and software for such correlative and statistical studies are well known in the art.

It is further contemplated that the discoveries of this invention can be used in monitoring and evaluating treatment protocols in subjects with musculoskeletal, arthritic and/or joint diseases and disorders as described herein. Thus, in further embodiments, the present invention provides a method of monitoring the therapeutic efficacy of a treatment regimen for a musculoskeletal, arthritic or joint disorder in a subject, comprising: detecting, in a sample from the subject, the amount of D-aspartate and/or an advanced glycation end product, either as an absolute amount in the sample or as a proportion of a total amount of a joint tissue molecule in the sample, over time prior to and/or during the treatment regimen, whereby a decrease in the amount of D-aspartate and/or advanced glycation end products after the onset of the treatment regimen and/or over time during the treatment regimen indicates therapeutic efficacy of the treatment regimen.

Further provided is a method of identifying an effective treatment regimen for a musculoskeletal, arthritic or joint disorder in a subject, comprising: detecting, in a sample from the subject, the amount of D-aspartate and/or an advanced glycation end product, either as an absolute amount in the sample or as a proportion of a total amount of a joint tissue molecule in the sample, prior to and/or over time during the treatment regimen, whereby a decrease in the amount of D-aspartate and/or advanced glycation end product after the onset of the treatment regimen and/or over time during the treatment regimen identifies an effective treatment regimen.

In the methods of this invention involving the monitoring of efficacy of treatment or identifying an effective treatment regimen as described herein, the amount of D-aspartate and/or advanced glycation end product measured can be an absolute amount or it can be a proportion of a total amount in a sample of a subject of this invention. A decrease in an absolute amount or in a proportion of a total amount in a subject as compared to an absolute amount or a proportion of a total amount in a control subject of this invention can be any decrease determined according to the methods of this invention to be relevant.

Thus, in some embodiments the decrease can be any value that is less than the value of the control and in other embodiments, the decrease can be any decrease beyond a threshold value. For example, in some embodiments, for a decrease to be indicative of efficacy, the decrease can be at least 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250 or 300% less than the value of the control. It is further contemplated that in some embodiments, a proportion of a molecule containing D-aspartate and/or an advanced glycation end product in a total amount of the molecule that is indicative of efficacy can be described as a decrease in a percentage of the total amount of the molecule being measured. Thus, for example, in a sample containing a molecule, a proportion of the total amount of the molecule that contains D-aspartate and/or an advanced glycation end product can be decreased in an amount of 0.5, 1, 2, 3, 4, 5, 6, 7, 8, 9, 10, 15, 20, 25, 30, 35, 40, 45, 50, 55, 60, 65, 70, 75, 80, 85, 90, 95, 98, 99 or 100% of the total amount of the molecule as compared to the control and be indicative of efficacy of a treatment or therapy protocol or regimen.

The present invention will now be described with reference to the following examples. It should be appreciated that these examples are for the purposes of illustrating aspects of the present invention, and do not limit the scope of the invention as defined by the claims.

### EXAMPLES

### Example 1

### Protein Standards for Aspartate Assays

Type II collagen, and aggrecan (A1D1 and A1D6 fractions) will be purified from human cartilage. The content of D-Asp and L-Asp in each protein preparation will be quantified by established HPLC methods using commercially available highly purified D-Asp and L-Asp for HPLC peak identification and quantification. The D-Asp content of these molecules will be expressed as a fraction of total Asp (D+L Asp). These proteins preparations will serve as the gold standards for quantifying the fraction of D-Asp in tissue and body fluids by immunoassays, such as ELISA.

### Example 2

### Enzyme Linked Immunosorbent Assays (ELISA) to Quantify the D-Asp Content of Type II Collagen Fragments and Aggrecan Fragments

ELISA assays will be developed to detect D-Asp in fragments of type II collagen and aggrecan in synovial fluid, serum and urine. The content of D- and L-Asp in the particular molecule will be determined with a detection system using commercially available polyclonal D- and L-Asp antibodies. Standards prepared in Example 1 will be used to quantify total D- and L-Asp content of the experimental samples. The types of collagen II and aggrecan fragments to be captured are as follows:

a) Type II collagen fragments will be captured with type IX collagen. Type IX collagen binds to the N- and C-telopeptides of collagen II and not collagen I. Preliminary results show high levels of collagen II fragments in the urine and measurable D-Asp in this fraction in OA subjects when this method of fragment capture is used.

b) A C-terminal type II collagen fragment that contains aspartate will be captured with an antibody to the (₁₂₂₇EKGPDP₁₂₂₅) epitope. This epitope is stable, survives to urine, and forms the basis of the current Cartilaps assay by Nordic Bioscience. An ELISA assay will be developed to measure the D- and L-Asp isomers within this epitope.

c) Type II collagen (prepared in Example 1) will be cleaved with cyanogen bromide and fragments separated by SDS-PAGE gel electrophoresis. Immunoblots will be performed to identify the specific D-Asp containing fragments. The fragments will be identified based upon well-established knowledge of mobility of the various type II collagen CNBr fragments in a gel. Monoclonal antibodies to these fragments will be developed for use in ELISA assays.

d) The fragments of aggrecan that bind hyaluronan will be captured with purified hyaluronan. The hyaluronan-binding domain of aggrecan is the G1 domain that has been shown to possess the highest quantity of D-Asp in cartilage. To measure D-Asp of the protein core of aggrecan, the sample to be assayed will be pre-treated with chondroitinase.

### Example 3

### Biomarker Dating

It is currently impossible to reliably distinguish an individual OA patient on the basis of a single biomarker at a single time point. Certain biomarkers have been identified to be associated with OA, including serum cartilage oligomeric matrix protein (COMP), serum hyaluronan, and various epitopes of type II collagen. The present invention provides a unique strategy that is a refinement of current OA biomarker methods that improves upon the predictive capability of current OA biomarkers. This refinement is based upon measuring the fraction of D-aspartate in select joint tissue molecules found in body fluids, in particular, type II collagen, and aggrecan.

Amino acids exist in native proteins as the L-configurational optical isomer. The L-isomer is converted to the biologically uncommon D-isomer by a spontaneous process (racemization) that is dependent on time, temperature, and to a lesser extent pH. Although in general, racemization is a very slow process, aspartate is one of the ‘fastest’ racemizing amino acids; this enables its detection in proteins that are not renewed or have a slow turnover rate. A protein-repair enzyme exists that methylates racemized aspartyl residues in age-damaged proteins. This repair methyltransferase operates intracellularly to prevent the accumulation of intracellular racemized aspartyl residues. However, extracellular matrix proteins are sequestered from the effects of this enzyme. In tooth dentin, where collagen is considered to be completely stable, racemization of aspartate closely follows first order kinetics with a rate constant of 0.3×10⁻³ per year. Racemization of aspartate is also detectable in the two joint tissue molecules in which it has been studied, collagen and aggrecan. The quantification of D-aspartate in these joint tissue molecules has revealed the presence of distinct pools of molecules with different turnover rates ranging from 100-400 years for collagen and from 3-25 years for aggrecan. The fraction of D-Asp in the fragments derived from these molecules present in the serum, urine and synovial fluid reflects the degree of catabolism of the oldest pool of joint tissue molecules. HPLC methods and immunoassays are used to measure the fractional levels of D-Asp in select joint tissue molecules in serum, urine and synovial fluid from OA and non-OA subjects. Quantification of the oldest type II collagen and aggrecan fragments in body fluids is expected to allow for better discrimination of an OA subject from a non-OA subject than is possible with currently available OA biomarkers. This refinement of current biomarker technology is expected to yield valuable insights into the contribution of catabolic processes (high biomarker level with a high D-Asp content) versus anabolic processes (high biomarker level due to high turnover state but with a relatively low D-Asp content) to the level of a biomarker in an OA subject. The technique of quantifying the D-aspartate content of molecules, such as joint tissue molecules, is referred to herein as “biomarker dating.”

‘Biomarker dating’ of circulating molecules in OA and control subjects. The different D-Asp containing cartilage biomarkers will be measured in urine, serum and in a subset of synovial fluids from OA subjects and controls by ELISA methods developed in Specific Aim 2. The validation in these cohorts will establish the utility and feasibility of the biomarker dating technique. The success of this endeavor would provide an exciting set of biomarker tools for subsequent analyses in the highly characterized samples obtained through the OA Initiative.

Through collaboration with Dr. Joanne Jordan, 800 serum samples from a cohort of individuals participating in a prospective population based epidemiological study of OA of the hand, knee and hip in Johnston County, N.C. are available to be tested in the assays of this invention. The COMP levels in these subjects have previously been quantified. The 800 subjects represent ¼ of the total ascertained population. These 800 subjects have been randomly selected to provide a sample balanced on knee OA affection status, gender, race and age. This sample is therefore ideal as an initial means of assessing the utility of measurements of the D-Asp content of type II collagen and aggrecan to serve as biomarkers of OA. Moreover, this sample provides a means of evaluating the association of D-Asp in collagen and aggrecan with age, gender and race. An increase in circulating D-Asp with age is expected, as well as an increase due to OA secondary to heightened catabolism of older molecules released into SF and the circulation. For this reason, the statistical analyses of the association of D-Asp levels and OA will control for age. Results will further be analyzed by computation of z-scores based upon results in the 400 control samples.

A second group of samples available is being generated by the POP study (Predicting OA Progression). This is a prospective study of subjects (n=150) with knee OA who undergo bone scan, knee radiography, and serum and synovial fluid collection for assays of cartilage oligomeric matrix protein, and urine collection for potential other assays in the future. This study is unique for its simultaneous collection of serum and synovial fluid samples in knee OA subjects.

The preliminary validation of the biomarker dating strategy in these populations will establish the utility and feasibility for using this strategy in other populations such as the samples obtained through the OA Initiative. It is expected that the greater the elevation of the D-Asp fraction of a circulating biomarker, the greater the uncoupling of catabolism and anabolism with the shift toward catabolism. These studies will serve as a model system for investigating turnover of aged pools of OA related proteins and biomarkers. Thus, this strategy could be applied to other cartilage markers as well. The development of monoclonal antibodies to D- and L-Asp will facilitate the characterization of other D-Asp containing cartilage extracellular matrix molecules. To date, D-Asp has been only been quantified in collagen and aggrecan. One candidate for future studies includes link protein, which is another long-lived molecule of the cartilage extracellular matrix. Overall, this strategy represents a refinement of OA biomarker work based upon the inventive concept that quantification of catabolism of the oldest resident molecules in cartilage allows for better discrimination of OA subjects from non-OA subjects.

Cartilage turnover. Cartilage is composed of two major constituents, collagen II and proteoglycan [1]. Proteoglycans, entrapped in the collagen network, make up ˜40% of the dry weight of cartilage. The proteoglycan component plays a crucial role in the structure of cartilage by endowing the tissue with its ability to reversibly absorb loads [1], so-called compressive stiffness. Collagen II makes up ˜60% of the dry weight of cartilage and provides tensile stiffness and strength. Collagen architecture of normal articular cartilage consists of layers of flat ribbons parallel to the surface, vertical columns in the intermediate zone and a random meshwork in the deep zone [2].

The skeleton is non-static. Cartilage, like bone, is in a continual state of resorption and formation. In osteoarthritis, the physiological balance between extracellular matrix synthesis and degradation is altered in favor of degradation. This appears to be due to a cell-mediated upregulation of normal degradative processes in combination with the synthesis of poorly assembled matrix pools of molecules [3]. Cartilage matrix macromolecules are thought to be compartmentalized into multiple metabolic pools that turn over at different rates [4]. Turnover is an active process requiring viable cells and protein synthesis as demonstrated by the inhibition of turnover by freeze thawing or addition of cycloheximide to cartilage explants [5]. Turnover is most rapid in the vicinity of viable cells. The metabolism of proteoglycan has been shown to be much more dynamic in the matrix which surrounds the chondrocyte than in the interterritorial matrix which is further removed from the cells [6]. Turnover also varies with distance from the articular cartilage surface [7] and age [8]. Type II collagen is degraded by interstitial collagenases, a class of matrix metalloproteinases (MMPs) that possess the unique ability to cleave an intact triple helical collagen fibril. Damage to the fibrillar meshwork of cartilage is considered a serious and irreversible occurrence due to the slow rate of collagen turnover within cartilage. The catabolism of aggrecan is mediated by metalloproteinases and aggrecanases. The release of small molecular weight G1-bearing species of aggrecan is commonly interpreted as a final stage in chondrocyte-mediated proteoglycan metabolism [3].

Racemization of aspartate. Amino acids in proteins are subject to a variety of spontaneous degradative processes, including oxidation, glycation, deamidation, isomerization, and racemization [9]. Racemization is the process of converting the L-configurational optical isomer of an amino acid to the biologically uncommon D-form. Racemization occurs spontaneously and non-enzymatically but is dependent on time, temperature and pH conditions. The rate varies for different amino acid residues, with the most rapid racemization being found for aspartate, followed by alanine=glutamic acid>leucine=isoleucine [10]. Overall, L-Aspartate is one of the most unstable residues in proteins, being particularly susceptible to deamidation, and isomerization, as well as racemization reactions [9]. Because aspartate is one of the fastest racemizing amino acids, the D-form can be detected in living subjects in proteins which are not renewed or which have a slow turnover rate, including the following: cartilage matrix proteins—collagen and aggrecan; tooth enamel and dentin; crystallins of the eye lens; and proteins of the brain [11]. Because the racemization of L-aspartate to D-aspartate (D-Asp) is dependent on time, the accumulation of racemized D-Asp represents a manifestation of aging at a molecular level.

The accumulation of age-related molecular species depends on the rate constants for the inversion from the L- to the D-form, and on the protein turnover rate. Three different scenarios can be envisioned regarding the turnover of D-Asp containing molecules. Scenario 1: If the turnover is zero, the D-Asp form accumulates in the tissue. This circumstance is unfavorable for exploitation as a biomarker of OA since no D-Asp is contributed to body fluids. This circumstance is however favorable for archeological [12] and forensic medicine dating of biologic specimens. An example of such a circumstance is provided by the accumulation of D-Asp in tooth dentin and enamel which follows first order kinetics and which has been exploited for the purpose of tissue dating [13-15]. Scenario 2: If the turnover were very high compared with the inversion rate, little or no racemized species would be detectable in body fluids as no racemized species could accumulate. This too is unfavorable for biomarker studies. Scenario 3: Finally, a state between scenarios 1 and 2 can exist in which age-related species build up in a tissue, reaching a steady state reflecting the metabolic activity of a particular protein. Cartilage exhibits the latter behavior. Cartilage is unique with regard to the relatively slow turnover of its extracellular matrix, making it susceptible to the accumulation of non-enzymatic molecular alterations.

Both type II collagen and aggrecan possess sufficient aspartate residues in which to potentially monitor D-Asp formation. The aspartate contents of collagen and aggrecan core protein as a percentage of total amino acids are calculated to be 3.2% and 4.5%, respectively. The racemization of aspartate to D-Asp has been used in cartilage research to calculate the half-lives of cartilage collagen (range 100-400 years; mean 117 years) [11, 16], and aggrecan (range 3-25; 3 years for the A1D1 component and 25 years for the G1 domain) [17]. These studies demonstrate that both collagen and aggrecan exist in pools within cartilage with sufficiently long enough resident times to accumulate D-Asp enantiomeric forms. The accumulation of these forms during normal aging can be used in the detection of OA characterized by accelerated turnover and excess of catabolism. Preliminary studies have demonstrated that sufficient turnover of type II collagen occurs to allow for detection of D-Asp containing fragments in urine from OA subjects. Moreover, D-Asp containing species of aggrecan fragments can be measured in the circulation. Thus, the assessment of D-Asp isoforms of joint tissue molecules in body fluids provides a dynamic index of the rate at which cartilage is resorbed. This molecular alteration is used as a tag to survey and follow the catabolism of the oldest resident cartilage molecules. This ability to distinguish catabolism of aged joint tissue molecules allows for better overall discriminatory power between OA subjects and controls than is currently possible by assessing the total level of a joint tissue molecules or its fragment in a body fluid. D-Asp can also be a biomarker of more aggressive and progressive forms of OA.

Western blot studies. Western blot analyses showed that D-Asp was present in proteins extracted from cartilage and in purified type II collagen, as well as in proteins precipitated from blood, synovial fluid and urine. Protein extracts were prepared from human knee articular cartilage of a 57-year-old patient with bilateral knee OA using Trizol reagent following the manufacturer's protocol as described. Briefly, after the removal of the aqueous phase for RNA isolation, the DNA was precipitated from the sample and the remaining phenyl-ethanol supernatant was dialyzed against three changes of 0.1% SDS at 4° C. and spun at 10,000×g for 10 minutes. Proteins were precipitated from the cartilage dialysate, human serum (normal and OA), synovial fluid (OA) and urine (OA) with trichloroacetic acid. The protein pellets were resuspended in 2×SDS loading buffer and run on 4-20% gradient SDS gels, which were either stained with Coomassie Blue or blotted onto 0.2 μM nitrocellulose membrane. After blocking, the nitrocellulose membranes were incubated with a 1:200 dilution of the anti-D-Asp polyclonal antibody (MoBiTec Cat #1055GE) overnight at 4° C. An anti-rabbit IgG antibody conjugated to peroxidase was used for detection of the reactive proteins using enhanced chemiluminescence.

The presence of a primary (˜70 kDa) D-Asp containing protein was detected in blood, synovial fluid and urine from OA subjects. Moreover, OA serum yielded approximately two-fold more of the D-Asp immunoreactive species as compared to control (non-OA) human serum (equal portions analyzed). Other smaller but less prominent immunoreactive species were also present. Strong D-Asp immunoreactive species were detected in purified type II collagen (preparation of commercially available, pepsin digested type II collagen from Chondrex, Cat # 2005-1). A cartilage extract from joint tissue obtained at the time of arthroplasty contained D-Asp immunoreactive species that co-migrated with type II collagen. The extract also contained the ˜70 kDa band seen in body fluids. This result indicates that a non-collagenous protein in cartilage is highly immunoreactive to D-Asp. Of note, there were no D-Asp immunoreactive species detectable by Western blot in purified cartilage oligomeric matrix protein from cartilage, however subsequent experiments using HPLC have demonstrated 6.1% D-Asp in this preparation Western blots of casein have demonstrated no detectable D-Asp. This result demonstrates that not all proteins contain D-Asp immunoreactive species.

ELISA studies. ELISA analyses of body fluids show that D-Asp is present in both collagen and aggrecan fragments in urine and serum.

D-Asp in collagen fragments. An ELISA assay was developed that captures type II collagen fragments from urine (Table 1). These assays demonstrated large quantities of type II collagen fragments, and specifically C-telopeptide fragments, in the urine. Moreover, these studies demonstrated the presence of measurable D-Asp immunoreactive fragments in a urine sample from an OA subject and from a subject with severe relapsing polychondritis. Purified Sigma type collagen IX (Sigma, Cat# C3657) was coated overnight at 4° C. onto a 96 well Immulon 4 plate at a concentration of 10 μg/ml in 0.1M NaCarbonate/0.5M NaCl buffer at pH 7.0. This preparation of collagen IX represents a pepsin digested and acid soluble fraction from human placenta [22]. This preparation of collagen IX has no D-Asp immunoreactivity using the concentrations and conditions of these experiments. Collagen IX associates specifically with the surface of, and participates in the formation of, type II collagen fibrils. Distinct domains within collagen IX react with the N- and C-telopeptide domains of type II collagen [23]. The plate was blocked with 1% bovine serum albumin for 1 hour at 37° C. After the plate was washed three times with Tris buffered saline (TBS), samples were added and incubated overnight at 4° C. Plates were again washed three times in TBS and one of two different primary antibodies was added: anti-collagen II monoclonal antibody to the C-telopeptide of type II collagen (Neomarkers Clone 2B1.5; 0.5 μg/ml dilution of 1:400) or an anti-D-Asp polyclonal antibody (MoBiTec Cat # 1055GE; 1:2000 dilution). Plates were again incubated for 2 hours at 37° C. and washed, followed by the addition of an anti-mouse alkaline phosphatase antibody (Promega; 1:5000 dilution) or an anti-rabbit alkaline phosphatase antibody (Sigma; 1:1000 dilution) for 1 hour at 37° C. Secondary antibody was quantified by incubation with substrate, p-nitrophenyl phosphate (Sigma), in DEA buffer (1M diethanolamine, 0.126 mM MgCl₂ in water, pH 9.8) at 25° C. The absorbance at 405 nm was read on a Tecan plate reader after 30 minutes.

D-Asp in aggrecan G1 fragments. An ELISA assay was developed that captures the G1 fragments of aggrecan based upon binding to hyaluronan (Table 2). These fragments only react with the D-Asp antibody after sample treatment with chondroitinase to expose the core epitope. The addition of keratanase had little additional effect. Immunoreactivity diminished with increasing serum dilution and fell to background levels at and below dilutions of 1:16. This method has the advantage that hyaluronan has no aspartate to potentially confound the assay as it is a polysaccharide. Purified hyaluronan (Sigma H1876, grade III hyaluronan derived from human umbilical cord; 100 μg/ml) in 0.5M NaCarbonate/0.5M NaCl buffer pH 9.5 was used to coat a 96 well Immulon 4 plate overnight at 4° C. Plates were blocked with 1% bovine serum albumin for 1 hour at 37° C. Serum samples (100 μl) were added at a 1:2 dilution directly to the plate or were added after pretreatment with chondroitinase ABC (0.005 units) or chondroitinase ABC and keratanase (0.005 units) for 4 hours in 0.2M Tris acetate buffer pH 7.5. Samples were incubated overnight at 4° C., followed by washing in Tris buffered saline. The remainder of the procedure was as described above with the use of the anti-D-Asp primary antibody and the anti-rabbit secondary antibody.

Taken together, these results confirm the presence of D-Asp in type II collagen and aggrecan fragments and demonstrate the feasibility of measuring D-Asp containing species in body fluids by immunoassay. Both a competitive format and a direct antibody capture with anti-D-Asp antibodies can be used in the immunoassays of this invention.

Production and characterization of a novel monoclonal antibody to the G3 domain of cartilage aggrecan [24]. The objective of these studies was to produce monoclonal antibodies (mAbs) to the G3 domain of human aggrecan spanning amino acids 1778-2379 to elucidate the mechanisms involved in G3 processing during the post-translational modification of aggrecan. One of these mAbs, 5A5, was extensively characterized and determined to be of the IgG2b isotype. This mAb was shown to recognize, both by Western blot and ELISA, purified recombinant GST fusion proteins containing the G3 domain, in addition to native cartilage aggrecan from fetal bovine articular cartilage (A1D1 fraction) and Swarm rat chondrosarcoma (D1D1 fraction). Amino acid sequence analysis of peptides derived from the purified recombinant CS/G3 confirmed the presence of the 5A5 epitope in the G3 domain. These results indicate that mAb 5A5 specifically recognizes the G3 domain of aggrecan.

Synovial fluid biomarkers in acute knee injury in humans [25]. Subjects (n=20) with acute knee injury of less than six months duration were evaluated for this study. Anterior cruciate ligament injury (ACL) was present in six subjects, meniscal damage was evident in four subjects, and ten subjects had injuries of both ACL and meniscus. Chondral damage was quantified using a validated arthroscopic scoring system [26]. Undiluted synovial fluid was collected by arthrocentesis prior to arthroscopy. Chondroitin-6-sulfate (CS), keratan sulfate (KS) and hyaluronic acid (HA) were measured, as these had been shown previously to reflect cartilage metabolism and serve as diagnostic or prognostic markers of osteoarthritis. Total sulfated-glycosaminoglycan (s-GAG) was measured in a 1,9-dimethylene blue colorimetric dye-binding (DMMB) assay. A dramatic decrease in synovial fluid levels of CS, KS and s-GAG was observed with increasing chondral lesion size (CS p value 0.005; KS p value 0.08; s-GAG p value 0.02). There was no correlation of synovial fluid HA levels with chondral lesion size. These data showed a detectable change in cartilage metabolism within the first six months of symptomatic knee injury. This indicates that the screening of synovial fluid levels for these markers could be predictive of chondral lesion severity and aid in the decision to intervene surgically.

Effects of chronic exercise on serum and plasma biomarkers in arthritic patients undergoing an Arthritis Foundation certified aquatics training program [27]. This study was undertaken to investigate the effects of a 14-week aquatic exercise program on circulating biomarkers of cartilage and bone metabolism. In a within-subjects, repeated measures design, 15 subjects with musculoskeletal disease were assessed at study entry and after a 14 week aquatic exercise program for 1) disease status based on the WOMAC index, 2) aerobic endurance using a 12-minute walk test and 3) circulating biomarkers indicative of cartilage and bone metabolism. Biomarkers included hyaluronan, keratan sulfate, cartilage oligomeric matrix protein and bone alkaline phosphatase. To assess the stability of biomarker measures in a sedentary population, a total of 16 additional subjects without musculoskeletal disease were evaluated for biomarker levels before and after a 6-month interval. Significant improvement in functional capacity occurred in the exercise group assessed by 12-minute walk distance and WOMAC score. Circulating hyaluronan increased with exercise. There were no significant changes in any of the other biomarkers with chronic exercise. All four measures were remarkably stable over time in the sedentary group. These data constitute objective evidence of an exercise induced alteration in a circulating biomarker indicative of joint metabolism, demonstrating the feasibility of applying biochemical measures to the study of exercise as a therapeutic intervention for arthritis.

Synovial fluid biomarker analyses in total canine medial meniscectomy [28]. The effects of total medial meniscectomy on biomarkers were evaluated in synovial lavage fluid, measured serially at monthly intervals for three months. Four biomarkers were evaluated following canine meniscectomy: cartilage oligomeric matrix protein (COMP), keratan sulfate epitope (5D4), the 3B3(−) neoepitope of chondroitin sulfate, and the 3B3(+) chondroitinase-generated epitope of chondroitin sulfate. Meniscectomy led to statistically significant elevations of all four biomarkers, with levels peaking at four weeks. By 12 weeks, the level of the 5D4 epitope returned to pre-operative baseline levels, while the 12-week levels of COMP, 3B3(−) and 3B3(+) continued to remain elevated above baseline. Concentrations of these biomarkers in the unoperated knees did not change significantly from baseline. The levels of COMP and 3B3(−) relative to 3B3(+) remained constant in both operated and unoperated knees. In contrast, the level of 5D4 relative to 3B3(+) declined over time in the operated knee but remained constant in the unoperated knee. These results demonstrated a quantitative change in the molecular components of synovial fluid after meniscectomy as well as a qualitative change evinced by an alteration in the relative proportions of these epitopes.

Synovial fluid biomarker analyses in allograft reconstruction after canine meniscectomy [29]. The goal of this study was to evaluate whether reconstruction of the medial meniscus with a fresh allograft will prevent the rise in synovial fluid biomarkers observed following total meniscectomy. Twenty adult mongrel dogs underwent complete open medial meniscectomy of the right knee; the left knee was used as a contralateral control. Half the animals were randomly assigned to receive a fresh medial meniscal allograft, based on matching the weight of the donor and recipient animals. Quantitative measurements were made of four biomarkers from lavage synovial fluid: 5D4, 3B3(−), cartilage oligomeric matrix protein (COMP), and 3B3(+). Knee joint synovial fluid lavages were performed with 5 ml of physiologic saline preoperatively and at 12 weeks after surgery. Significant increases were observed in concentrations of 3B3(−), 3B3(+), and COMP at 12 weeks following meniscectomy, expressed as a ratio of experimental over control joints. No significant changes were observed in the concentration of these three markers in animals, which had received a meniscal allograft. No significant change was detected in 5D4 concentration with meniscectomy or allograft reconstruction. This study indicated that the concentrations of 3B3(−), 3B3(+), and COMP in the synovial fluid can serve as reproducible markers of meniscal injury or cartilage damage. Allograft reconstruction of the medial meniscus prevented the significant increases in the 3B3(−), 3B3(+), and COMP biomarkers observed with meniscectomy. This finding suggests a potential beneficial effect of allograft reconstruction on the health of the synovial joint.

Characterization of collagenase-1 and collagenase-3 in the guinea pig model of OA [30]. Competitive reverse transcription-polymerase chain reaction (RT-PCR) and immunohistochemistry were used to quantify mRNA and protein levels of collagenase-1 and -3 in medial and lateral tibial cartilage of knee joints in 2-month-old (no OA pathology) and 12-month-old (OA pathology) guinea pigs. The patterns of mRNA expression of collagenase-1 and -3 varied with the age of the animal and the compartment of the knee. Focal localization of collagenase-1 and -3 proteins to the extracellular matrix of OA lesion sites was found coincident with ¾-¼ collagen cleavage detected by monoclonal antibody 9A4. Collagenase-3 protein was also abundant throughout the medial tibial cartilage of 2-month-old animals. This represented the first description of bona fide collagenase-1 in a rodent species. The presence of active collagenase-1 and -3 at OA lesion sites is consistent with an important role for these enzymes in the cartilage degradation of OA in guinea pigs. The early expression of collagenase-3 in 2-month old medial tibial cartilage suggests a role for this enzyme in cartilage remodeling with growth and development or as an early molecular manifestation of OA.

Synovial fluid and serum biomarker analyses in a guinea pig model of OA [31-33]. The objective of this work was to evaluate guinea pig strains that might serve as age-matched controls for the OA-prone Hartley strain, and to distinguish pathological, OA-associated from age-related interactions of the major joint tissues. Two guinea pig strains (Hartley and Strain 13, 12 months of age) were evaluated for cartilage and bone pathology using semi-quantitative histological grading of knee joints and quantification of biomarkers including urinary excretion rates of hydroxylysyl-pyridinoline (HP) and lysyl-pyridinoline (LP) collagen crosslinks, serum osteocalcin, and synovial fluid levels of keratan sulfate (KS). Strain 13 had minimal to mild histological evidence of OA compared to Hartley strain. Moreover, Strain 13 had lower intra-articular proteoglycan turnover, and lower bone turnover. Levels of synovial fluid keratan sulfate were positively correlated with the severity of histological OA. Increasing subchondral bone thickness with age was characteristic of both Hartley and Strain 13, but Strain 13 possessed much thicker subchondral bone at the outset (2 months) compared to the Hartley. This study represents the first evidence of differential susceptibility to OA in guinea pigs. Comparison of these two strains of guinea pig has revealed that increased metabolism within the affected tissues, cartilage and bone, is associated with the development and progression of OA. Moreover, thicker subchondral bone prior to demonstrable chondropathy did not predispose the Strain 13 to more severe OA. In an extended longitudinal analysis of the OA prone Hartley strain, a steady worsening of histological knee was found from 4-12 months of age in association with a strong correlation of histological severity to synovial fluid concentrations of cartilage oligomeric matrix protein and keratan sulfate. Finally it was determined that reductions in normal collagen network birefringence corresponded to histological progression of OA in this model and that disruption of the collagen network was discernable prior to evidence of histological OA. Evidence for collagen network disruption corresponded to the appearance of the collagen neoepitope generated by collagenase (detected by monoclonal antibody 9A4 from Pfizer), which also appeared prior to histological evidence of OA.

Serum cartilage oligomeric matrix protein (COMP) studies. [34-39]. In these studies, serum cartilage oligomeric matrix protein (COMP) levels were measured in a radiographically defined population in rural North Carolina, a random sampling of the cohort of the Johnston County Osteoarthritis Project, a population based study of OA of hip and knee osteoarthritis (OA), to examine the potential utility of COMP as a diagnostic biomarker for knee OA [34]. A total of 291 samples were randomly selected for COMP analysis, 143 with radiographic knee OA (Kellgren-Lawrence grade≧2) and 148 controls free of knee and hip OA (Kellgren-Lawrence grade 0), evenly distributed by age and gender. COMP was quantified by competitive ELISA assay with monoclonal antibody 17-C10. Serum COMP was significantly elevated in the group aged 65 years and above (mean±std. dev. 1302.1±496.7 ng/ml) compared to the group younger than 65 years (1048.5±377.6 ng/ml, p=0.0001). Mean serum COMP levels of the OA group (1208.6±487.5 ng/ml) were significantly higher than levels of the control group (1061.8±370.6 ng/ml, p=0.0093). Serum COMP levels also increased significantly with severity of OA (p=0.0047) and number of knee and hip joints involved (p=0.0002). There was no significant difference in serum COMP by gender or obesity. These results demonstrated that in a population-based sample, serum COMP levels can distinguish an OA-affected from an unaffected subgroup, and can reflect disease severity and multiple joint involvement with OA. Further characterization of the control group, without radiographic evidence of knee or hip OA, revealed that subjects with clinical signs and symptoms of arthritis (especially with joint pain and hip-related variables), had higher serum COMP levels [35]. In the first OA-related biomarker study to include African-Americans [36], serum COMP levels were studied in a large, radiographically defined and ethnically diverse population-based sample consisting of 379 African-Americans and 390 Caucasians. COMP levels were found to be significantly higher in African-American women than in Caucasian women and in Caucasian men compared to Caucasian women, regardless of age, body mass index (BMI), and the presence or severity of radiographic knee or hip OA. This study documented ethnic and gender differences in serum COMP and has implications regarding the development and use of standards for this potential OA biomarker. In subsequent analyses, these differences were found to be largely explainable on the basis of ethnic differences among women in hormone replacement therapy (HRT) use. Current HRT use was associated with reduced levels of serum COMP in postmenopausal women irrespective of ethnicity [40]. Finally, serum COMP was evaluated as a prognostic marker of OA progression [38]. Subjects with progressive OA over three years were shown to have significantly higher COMP levels at baseline as well as at study end.

Validation of synovial fluid biomarkers as predictors of quantitative histological changes in the canine meniscectomy model of OA [41]. The purpose of this study was to document the histological changes present in the tibial plateaus 12 weeks after complete medial meniscectomy in dogs and to determine if synovial lavage fluid biomarker levels are predictive of the severity of joint damage. Twelve adult dogs underwent complete unilateral medial meniscectomy and synovial lavage fluid biomarker levels, including cartilage oligomeric matrix protein (COMP), keratan sulfate (5D4), 3B3(−), and 3B3(+), were measured serially at four-week intervals. The dogs were euthanized 12 weeks after surgery and each medial and lateral tibial plateau from the meniscectomized and contralateral knees was graded histologically. Histological data were analyzed using principal components analysis, which resulted in four factors that explained 70% of the variation in the data. Factor 2 (weighted most heavily by subchondral bone thickness) and Factor 3 (representative of articular cartilage damage) were significantly affected by compartmental site (p<0.01 for both). Both of these factors were highest in the medial tibial plateau of the meniscectomized knee, and Factor 3 was significantly higher in this site than in the medial tibial plateau of the contralateral knee (p<0.01). Peak levels of all four synovial lavage fluid biomarkers occurred at 4 weeks post meniscectomy and 4-week minus baseline levels of all biomarkers were significantly correlated with the Factor 3 scores. This study demonstrates that significant articular cartilage damage occurs relatively quickly following complete medial meniscectomy in dogs and establishes the content and criterion validity for these synovial fluid lavage biomarkers as surrogate measures of articular cartilage damage.

The validation of urea measures as a robust method of quantifying and correcting for the dilutional effect on synovial fluid biomarkers due to joint lavage or inflammation [42]. The goal of this study was to develop a method to correct for the unknown dilution of synovial fluid that occurs during lavage of the joint. Joint fluids were obtained from a total of 55 canine joints. Joint fluid was aspirated directly from 41 normal joints and by lavage from 10 normal joints. Acute joint injury was induced in four joints by intra-articular chymopapain injection. Serum and joint fluid urea were measured along with joint fluid concentrations of glucose, lactate, cartilage oligomeric matrix protein (COMP), and keratan sulfate (KS). Joint fluid urea concentrations were directly proportional to serum urea concentrations throughout a wide range of concentrations in normal joints. From this relationship, the dilution factor introduced by lavage was determined. This method was applied to quantify biomarker concentrations in synovial lavage fluid and was found to successfully correct for lavage-induced dilution of glucose, lactate, COMP, and KS in the joint fluid to levels equivalent to samples aspirated directly. In the context of joint effusion induced by chymopapain treatment, urea concentrations continued to be proportional to serum concentrations, but were much lower, enabling an estimation of the change in the volume of distribution (V_{d}) of a marker due to a change in joint water content in the setting of inflammation characterized by effusion. Lactate and KS rose markedly in response to chymopapain. After adjustment for the V_{d}, the concentration of glucose in the chymopapain injected joints did not change. Urea provides a robust method of quantifying and correcting for the dilution of synovial fluid due to joint lavage or inflammation. This method is potentially applicable to surrogate marker studies in human arthritis.

Serum Hyaluronic Acid (HA) Levels [43] and serum C-Reactive Protein (CRP) levels [44] and radiographic knee OA in African-Americans and Caucasians.

These analyses included 761 individuals with mean age (SD) of 61.9 (10.3) years, 48.9% African-Americans, and 41.9% males. Serum In HA was higher in Caucasians and in men (p<0.009), moderately correlated with age (r=0.343, p<0.0001), and weakly correlated with BMI (r=0.060, p=0.096). Ln HA was strongly positively associated with all definitions of radiographic OA (p<0.0001), with In HA increasing with severity of OA affection status. In contrast, associations between In CRP and radiographic OA were not independent of BMI. In separate models adjusted for BMI and other covariates, In CRP was independently associated only with ethnicity, BMI, and chronic pulmonary disease, and was not associated with OA presence or severity.

Preparation of protein standards for D-Asp assays. Surgical waste tissues taken at the time of joint arthroplasty for OA will be obtained. Cartilage specimens from approximately ten subjects over 65 will be pooled to provide a large amount of characterized protein. Cartilage will be cleaned of all adhering soft tissue, including surface perichondria and grossly calcified regions discarded. Tissue will be shredded and minced then extracted with 4M guanidine HCl for 24 hours. The soluble fraction will be used for aggrecan isolation. The insoluble fraction will be used for type II collagen isolation.

Collagen isolation. Type II collagen will be solubilized from the cartilage residue by the procedure of Miller [45], digesting the cartilage residue with pepsin at 4° C. in 3% acetic acid. Type II collagen will be purified by precipitation sequentially at acid and neutral pH as described [45] and shown to yield a single peak of pure alpha1(II) collagen [46].

Aggrecan (full monomer and G1 domain) isolation. The A1D1 (full aggrecan monomer) and the A1D6 (aggrecan G1 and link protein) fractions of cartilage will be prepared from the soluble fraction resulting from 4M guanidine HCl extraction. For the A1 fraction an associative gradient will be run using CsCl (˜1.65 g/ml) to achieve a starting density of 1.5 g/ml. Ultra-centrifugation will be performed for 36 hours at 4° C., 36,000 rpm, with no brake. The A1 fraction will be harvested by inserting a spinal needle into the bottom of the tube and drawing off ⅓ of the volume. The A1 fraction will be diluted 1:1 with 2× extraction buffer (e.g., 8M guanidine HCl). CsCl will be added to a starting density of 1.5 g/ml and ultra-centrifugation will be performed for 36 hours, 4° C., 36,000 rpm, no brake. The tubes will be frozen upright and cut with a sharp razor blade into six equal portions. The bottom portion of the tube will be considered to represent an A1D1 fraction (full length aggrecan monomers) and the top portion the A1D6 fraction (the G1 domain of aggrecan). The resulting protein fractions will be dialyzed against water, frozen and lyophilized. For use in D-Asp assays, these fractions will be reconstituted in 0.1M Tris Acetate, pH 8 and treated with chondroitinase ABC using 1 unit of chondroitinase for 10 mg of aggrecan A1D1 or A1D6. Samples will be incubated for 6 hours, 37° C. The adequacy of the digestion will be monitored by a rise in the absorbance at 232 nm. After digestion, the preparations will be dialyzed against water for 24 hours, 4° C., then lyophilized and finally reconstituted on 0.1M Na phosphate pH 7.0 buffer or 0.1M Na carbonate buffer for plate coating and stored at 4° C. The A1D6 fraction will be prepared by standard methods [59]. Monoclonal antibodies to chondroitin 4-sulfate and chonroitin-6 sulfate (e.g., 2B6 and 3B3 monoclonal antibodies), as well as to keratin sulfate (e.g., 5D4 monoclonal antibody) will be used, alone and in combination, to capture the majority of aggrecan fragments for development of ELISA assays.

HPLC methods. The D-Asp content of cartilage collagen has been successfully measured by HPLC [11, 16]. The D-Asp content of aggrecan has also been successfully measured by HPLC [17, 47]. The percentage of D-Asp in cartilage collagen and aggrecan will be determined by high performance liquid chromatography (HPLC) according to methods of Verzijl [16] (with modification). Standard materials for all assays will be calibrated for D-Asp content (HPLC method of Verzijl et al) [16]. Purified collagen II, aggrecan, and COMP will be acid hydrolyzed in 6M HCl at 100° C. for 4 hours. After evaporation to dryness, the hydrolysates will be reconstituted in 0.1M sodium borate buffer (pH 9.5). The resulting free D-Asp in the hydrolysates will be derivatized to a fluorescent compound by the addition of 0.03M o-phthalialdehyde/0.06M N-acetyl-L-cysteine in 0.1M sodium borate (ph 9.5). The subsequent derivatives will be separated by HPLC using a reversed-phase C18 column (150 mm×4.6 mm, 5 μm particle size) with a two step, two solvent, gradient elution (solvent A: 50 mM sodium acetate, pH 5.9 and solvent B: methanol). The gradient profile will be as follows: 5 minutes isocratic at 93% A, 7% B; linear increase in B over 5 minutes to 20% A, 80% B followed by 10 minutes isocratic at this ratio; equilibration for 10 minutes at 93% A, 7% B before the next injection. The resulting peaks will be quantified fluorometrically (excitation 340 nm, emission 440 nm) by comparison to known amounts of pure D-Asp commercially available from Fluka.

ELISA Assays for D-Aspartate. The quantification of Asp racemization in proteins is traditionally accomplished by chromatographic analysis. This method has the limitation that it does not provide information on racemization at an individual site within a protein. Moreover, the process of protein hydrolysis can produce additional D-enantiomers. For these reasons and for ease of analysis, ELISA based measurements will be developed for quantifying D-Asp content of joint tissue molecules and their fragments in body fluids.

The initial ELISA assays will be carried out using anti-Asp polyclonal antibodies to the D-form and to the (D-+L-) forms available through MoBiTec (Gottingen, Germany, Cat # 1055GE and #1011GE). This anti-D-Asp antibody was raised in rabbits after immunization with the conjugate, D-aspartic acid-glutaraldehyde. The specificity of the MoBiTec polyclonal anti-D-Asp polyclonal antibody has been certified by the company to be specific for the D-isomer of aspartate. Cross reactivity of the MoBiTec anti-conjugated D-Asp antibody was tested by ELISA competition experiments. The cross reactivity ratio was defined as the concentration of pure antigen (D-Asp-G-BSA)/the concentration of various compounds at half displacement that might potentially cross react. For D-Asp-G-BSA, the ratio was 1; for L-Asp-G-BSA, the ratio was 1:10,000; for D-Asp, the ratio was 1:>100,000 and for L-Asp, the ratio was 1:>100,000. The antibody is 10,000 fold specific for D-Asp over L-Asp and >50,000 fold specific for D-Asp over N-Methyl-D-Aspartic Acid. The anti-(D-+L-)-Asp antibody reacts to conjugated aspartate 100,000 fold over free aspartate. The standard curves for each ELISA assay will be fitted using linear regression (R²>0.99) (KaleidaGraph 3.0, Synergy Software, Reading, Pa., USA). All samples and standards will be measured in duplicate and at multiple dilutions. The amount of epitope in a sample will be calculated from the absorbance readings falling within the linear portion of the standard curve and expressed in ng/ml.

Type II collagen fragments will be captured with Sigma type IX collagen as described herein. Type IX collagen binds to the N- and C-telopeptides of collagen II but not collagen I. A portion of the N- and C-telopeptides of type II collagen is removed by pepsin digestion necessary for collagen isolation. Nevertheless, preliminary results show high binding activity for a commercially available pure type II collagen to Sigma collagen IX, high levels of collagen II fragments in the urine, and measurable D-Asp in this fraction in OA subjects.

The fragments of aggrecan that bind hyaluronan will be captured with purified hyaluronan as described herein. The hyaluronan binding domain of aggrecan is the G1 domain that has been shown to possess the highest quantity of D-Asp in cartilage. To measure D-Asp of the protein core, the sample to be assayed will be pre-treated with chondroitinase as described herein. The glycosaminoglycan content of the specimen will be quantified independently using the dye 1,9-dimethylmethylene blue [48]. The dye will be used at a concentration of 16 mg/L in formate buffer, pH 3.5. Forty ml of sample or standard (diluted in PBS, pH 7.2) will be added to wells of a microtiter plate with 250 ml of dye reagent and the absorbance at 530 nm and 600 nm will be read using a microplate reader. A negative absorbance change is obtained at 600 nm and a positive absorbance change is given at 530 nm. The total change in absorbance is determined as the sum of the two changes in absorbance. Chondroitin sulfate from shark cartilage will be used as a standard between 5 and 50 mg/ml.

The C-terminal type II collagen fragment (₁₂₂₇EKGPDP₁₂₂₅) has been shown to survive to urine and to be concentrated roughly 30-fold in urine over serum. This epitope forms the basis of the current Cartilaps assay whose levels have shown good correlation with OA [49]. An ELISA assay will be developed to measure the D- and L-Asp isomers within this epitope.

In addition to the above strategies, D-Asp containing peptides within type II collagen will be identified to form the basis for the development of other possible specific and OA relevant assays of D-Asp in body fluids. Specific cleavage at methionyl residues using cyanogen bromide, followed by resolution of the peptides by SDS-PAGE, produces a map that is unique for the collagen chain under study [50, 51]. A total of 3 mg of type II collagen, prepared as described herein, will be dissolved in 1 ml of 70% formic acid containing 50 mg of cyanogen bromide according to a previously published method [52]. Peptides of type II collagen will be identified by comparison with human type II CB peptide standards and published type II CB peptide maps [50]. Immunoblots will be performed as described herein to identify the specific D-Asp containing fragments of type II collagen. Synthetic peptides corresponding to the D-Asp containing CNBr fragments will be synthesized by SynPep to contain aspartate in the D-configuration. Monoclonal antibodies to these peptides will be developed in collaboration with a custom antibody biotech company (Spring Valley Laboratories) for use in ELISA assays.

The strong 70 KDa species from cartilage, identified by immunoblot to contain D-Asp will also be characterized. A Western blot of a cartilage extract will be carried out as described herein, with transfer to PVDF membrane. Adjacent lanes will be immunostained and the band of interest cut out and subjected to N-terminal sequencing, analysis of % D-Asp content, tryptic digests and mass spectrometry fingerprinting, according to protocols well known in the art.

Monoclonal antibodies to three epitopes, D-Asp, L-Asp, and the collagen II epitope EKGDPD, will be produced. This aspartate containing epitope is specific for collagen, is highly stable, and survives to urine. The amino acids and the synthetic peptide will be conjugated to KLH. Injections and antibody will be made in mice for monoclonal antibody production. Cartilage extracts of collagen and aggrecan from cadaveric young (less than 20 years old) and old tissue will be purified to serve as negative and positive controls for antibody screening purposes. The antibodies to D-Asp and L-Asp will be checked for cross-reactivity and selected for binding activity to the desired optical isomer using commercially available pure optical isomers of D- and L-aspartate (Fluka cat #11200 and #11189, respectively). The D-Asp monoclonal antibody will be screened to select specificity to D-Asp and lack of cross-reactivity to L-Asp or other amino acids. The monoclonal antibody to be used in ELISA assays will also be chosen on the basis of the ability to detect D-Asp in situ (in a protein versus the free amino acid) based upon reactivity to the D-Asp containing standards described herein. The utility of D-Asp to serve as a biomarker of OA will be tested with this ELISA based assay by quantifying the content of total D-Asp and D/L ratios in OA and control samples described herein. Hybridomas will be grown in RPMI complete supplemented with 10% BSA. After hybridomas are grown to confluence in 60 mm tissue culture dishes, they will be transferred to 350 ml concentrator chambers (Integra CL 350, INTEGRA Biosciences, Inc.) in order to achieve high concentration of monoclonal antibodies. Antibodies will be collected for at least two weeks.

The monoclonal antibody screening strategy will be three-tiered. The antibodies will be screened initially by ELISA against D-Asp-BSA conjugates and L-Asp=BSA conjugates. The antibodies will then be screened for reactivity to the synthetic peptide D-Asp or L-Asp containing peptide EKGPDP. Finally, the antibodies will be screened further against the HPLC characterized and D-Asp-containing cartilage/collagen standards described herein.

Measurement of D-Asp and L-Asp Using the SELDI Platform

In addition to HPLC and ELISA methods to measure D- and L-Asp as described herein, methods of differentiation of these isomers will also be employed based upon the SELDI (Ciphergen) platform. Monoclonal antibodies to D- or L-Asp will be coupled to a commercially available chip (Ciphergen). A sample, such as tissue of body fluid will be incubated for one hour at 25° C. with the chip. The chip will be washed with saline and the bound antigen (D- or L-Asp) will be quantified by mass spectrometry.

Biomarker dating of circulating molecules in OA and control subjects. Two patient populations are available for the studies described herein, the Johnston County Osteoarthritis Cohort (JOCO) and the Prediction of OA Progression (POP) study cohort. These studies define OA radiographically. The association of D-Asp with OA in these cohorts will establish justification for use in the samples generated in the OA Initiative which will include the more sensitive means of detecting OA, namely by MRI.

OA patient populations. JOCO: Through a collaboration with Dr. Joanne Jordan at the University of North Carolina at Chapel Hill, serum and urine samples are available in which to evaluate the utility of ‘biomarker dating’ in OA and control samples. A random sampling of this cohort (800 of the total 3200 participants) will be used, balanced on OA affection status, gender, age, and ethnicity, to evaluate a number of other biomarkers in the serum: cartilage oligomeric matrix protein [34-36], hyaluronan [43], C-reactive protein [44], keratan sulfate and osteocalcin. The Johnston County OA Project is the only population-based prospective longitudinal study of radiographic, functional, and psychosocial outcomes of knee and hip OA in African-Americans and Caucasians [53].

Johnston County has a population of about 100,000 and a rural area of about 800 square miles. A majority of residents (66%) live in completely rural areas, with the remainder in small towns. African-American residents constitute 20% of the population and residents 60 years of age or older, 17%. Baseline, cross-sectional data from this study have documented ethnic differences in multiple radiographic, symptomatic, and functional features of hip and knee OA, as well in the effect of risk factors for OA, such as obesity and diet [53-57]. Funding for the Johnston County OA Project infrastructure and recruitment is made possible jointly by the Centers for Disease Control and Prevention (CDC) and the National Institutes of Health (NIH). A baseline evaluation of 3200 participants was completed between May 1991, when data collection for the project began, and Dec. 31, 1997. The follow-up examination of these participants began in the fall of 1998 and is now complete. Each individual has undergone clinical evaluation (including hand examination for OA), radiographic assessment of knees and hips, and serum sampling at the baseline and follow-up examinations. Sera from 800 individuals are available from the initial evaluation. These are the samples assayed for other OA biomarkers as described herein. Urine samples are available from the entire cohort starting with the second evaluation.

POP: The POP study, funded by the NIH/NIAMS, is a prospective study with anticipated involvement of 150 subjects with knee OA evaluated by bone scan and radiograph at baseline and three years. This study is unique in two particular ways: first, attempts are made to collect synovial fluid from both knees of every subject; and second, a small pilot study (n=20) is included which allows collection of blood and urine samples for studies of diurnal variation of a marker (samples collected on the Duke GCRC prior to arising from bed and at 1, 4 and 8 hours after arising). Synovial fluid collection has been successful in approximately 95% of knees. When synovial fluid is not available by direct aspiration, the knee is injected with 10 ml (preservative free) sterile saline and aspirated. Synovial fluid concentrations of urea have been shown to serve as the basis for correcting for dilutional effects of lavage [42]. Urea concentrations can be measured on as little as 2 μl of synovial fluid with great sensitivity and with high precision, using a CMA600 microdialysis analyzer (CMA Microdialysis, Solna, Sweden).

In addition to OA status and severity, three main sociodemographic characteristics will be evaluated: age, gender, and race. A great deal of information has been collected on the individuals in the Johnston County OA Project that can be controlled for in the present invention if needed. Examples of potential confounders include socioeconomic status, body mass index, previous joint injury or surgery, general health status, occupation and home activities, and cigarette smoking. As noted in past studies, the presence of synovitis increased serum COMP levels. Information on joint tenderness and effusion is elicited by clinical examination in the Johnston County OA Project that will allow for an evaluation of the potential effects of synovitis on biomarker data in samples from this cohort.

The relationship between outcomes and biomarker values, and differences by age, gender, and ethnic group will be examined. A stratified random selection of cases and controls (400 each) has been used, to give a sample balanced on gender and age groups (range of age groups: 45-54, 55-64, 65 and above) and race (half Caucasian and half African American).

For a study with 80% power to detect a small difference between OA and normal subjects (effect size 0.3), it is estimated that an analysis of 200 control subjects without radiographic knee or hip OA, and 200 subjects with radiographic knee OA will be needed. Previous results with cartilage oligomeric matrix protein revealed that a total of 291 subjects (half cases and half control) was sufficient to show significant differences in the two populations. The effect size in these preliminary studies for COMP was 0.57 and therefore much greater than the conservative estimate. The sample size for the D-Asp studies is almost three times this so it is anticipated that it will be adequate to detect a difference in OA and non-OA groups.

To define OA, AP radiographs and sunrise views of the patellae were obtained at the baseline JOCO examinations. Semi-flexed PA knee radiographs and sunrise views of the patellae are obtained on all individuals in the Johnston County Osteoarthritis Project starting with the second examination and on all the POP study participants. The films are read using the Kellgren-Lawrence atlas for overall radiographic grade and the knee atlas from the Baltimore Longitudinal Study of Aging for scoring of individual radiographic features [58].

The D-Asp content of collagen and aggrecan fragments in serum, urine, and synovial fluid will be measured. D-Asp will be quantified as a ratio of total Asp content of synovial fluid to provide a global measure of catabolism of aged protein. All analyses will be performed using SAS and S-plus. Overall analyses will be done as well as analyses stratified by ethnic group. General linear models methodology, namely Analysis of Variance (ANOVA) models and Analysis of Covariance models, will be used to assess the relationship between the OA status and the mean baseline biomarker level. ANOVA models with log transformation where appropriate and as suggested by preliminary data, will be used to assess differences in means of biomarker levels between the OA status groups, adjusting for age group, gender, race, and obesity status. Adjusted (least squares) means of biomarker levels and the corresponding 95% confidence intervals will be provided for each of the OA status groups.

Although the primary ANOVA analyses will focus on the comparison of the two groups of interest (i.e., radiographically OA affected subjects and unaffected subjects), secondary analysis will separate the affected subjects with respect to number of joints with OA, unilateral/bilateral involvement, type of joint affected (knee only or knee and hip), and the Kellgren-Lawrence grade.

### Example 4

### D-Aspartate and Advanced Glycation End Products

A. Urinary % D-Aspartate (D-aspartate/total D-L aspartate) was shown to be strongly correlated with age. Levels of L-Aspartate and D-Aspartate were measured using the HPLC method of Hashimoto et al. with some modifications [61]. In a bivariate fit of % D-Asp by age, the linear fit of % D-Asp was 5.7129781+(0.0264933*Age), with a correlation coefficient r=0.62, and p=0.01.

B. Urinary D-aspartate concentration was measured by HPLC and normalized to urine creatinine in a female 47 yr old OA patient and compared to an age matched female 47 yr old non-OA control and a non-OA male control of roughly similar age (51 yrs old). The female OA patient sample showed a two-fold elevation in urinary D-aspartate level compared to the controls.

C. Levels of L-aspartate and D-aspartate were measured by HPLC in the urine from an OA patient and a non-OA age matched control. In the resulting chromatogram, with identical internal standard peaks, a much larger D-aspartate peak was seen in the urine of the OA patient compared to the non-OA age matched control.

D. Urinary D-aspartate levels (measured by HPLC) correlate with urinary uTIINE levels (type II collagen neoepitope indicative of articular cartilage degradation) in a patient with relapsing polychondritis characterized by severe cartilage destruction. The uTIINE test measures a collagen II fragment in urine that is derived from articular cartilage and correlated with disease severity and response to treatment [62]. The concentration of D-aspartate normalized to creatinine yielded a pattern similar to that of the collagen biomarker (uTIINE) indicative of collagen destruction.

E. Standardized ELISA assays for the presence of D-aspartate and L-aspartate in a peptide or protein. Inhibition ELISA assays have been developed to quantify the amount of D-Asp or L-Asp in a given sample. The assay is able to detect D-Asp and L-Asp as demonstrated by reactivity of the standard curves ranging from 5 μg/ml to 0 μg/ml at 2-fold dilutions for both D-Asp and L-Asp.

F. Purified COMP, purified collagen II from articular cartilage and purified collagen V contain HPLC-measurable quantities of D-aspartate: COMP: 6.1% D-aspartate; collagen II (Chondrex #2005-1): 5.1% D-aspartate; and collagen V (Sigma C2657): 7.4% D-aspartate.

G. Purified collagens I, II and V and bovine serum albumin were shown to contain D-aspartate based upon Western blot analysis with antibodies to D-aspartate. Proteins were detected by both alkaline phosphatase and NBT/BCIP, as well as by horseradish peroxidase and enhanced chemiluminescence.

H. Purified collagens I, II and V, bovine serum albumin and bovine nasal cartilage extract were shown to contain L-aspartate based upon Western blot analysis with antibodies to L-aspartate. Proteins were detected by both alkaline phosphatase and NBT/BCIP, as well as by horseradish peroxidase and enhanced chemiluminescence.

I. Detectable amounts of an advanced glycation end product (in this case, pentosidine) were shown to be present in COMP (5 μg) from articular cartilage by Western blot with antibodies to advanced glycation end products (including pentosidine).

Throughout this application, various patents, patent publications and non-patent publications are referenced. The disclosures of these patents, patent publications and non-patent publications in their entireties are incorporated by reference herein into this application in order to provide written support for the embodiments of this invention by reference to the teachings of the cited document and/or to more fully describe the state of the art to which this invention pertains.

The foregoing is illustrative of the present invention, and is not to be construed as limiting thereof. The invention is defined by the following claims, with equivalents of the claims to be included therein.

### REFERENCES

1. Kuettner, K., E.-M. Thonar, and M. Aydelotte, Modern aspects of articular cartilage biochemistry, in Cartilage Changes in Osteoarthritis, K. Brandt, Editor. 1990, Ciba Geigy. p. 3-11.
2. Hwang, W., et al., Collagen fibril structure of normal, aging, and osteoarthritic cartilage. J Pathol, 1992. 167: p. 425-433.
3. Quinn, T. M., et al., Physical and biological regulation of proteoglycan turnover around chondrocytes in cartilage explants. Implications for tissue degradation and repair. Ann N Y Acad Sci, 1999. 878: p. 420-41.
4. Gallagher, J. T., Concepts of metabolic pools in the metabolism of proteoglycans and hyaluronic acid. Biochem Soc Trans, 1977. 5(2): p. 402-10.
5. Handley, C., et al., Steady-state metabolism in cartilage explants, in Articular Cartilage Biochemistry, K. Kuettner, R. Schleyerbach, and V. Hascall, Editors. 1986, Raven Press: New York. p. 163-179.
6. Mok, S. S., et al., Aggrecan synthesized by mature bovine chondrocytes suspended in alginate. Identification of two distinct metabolic matrix pools. J Biol Chem, 1994. 269(52): p. 33021-7.
7. Maroudas, A., Glycosaminoglycan turn-over in articular cartilage. Phil Trans R Soc London—series B: Biological Sciences, 1975.271: p. 293-313.
8. Sandy, J. D., Plaas, A. H. K., Age-Related Changes in the Kinetics of Release of Proteoglycans from Normal Rabbit Cartilage Explants. Journal of Orthopaedic Research, 1986. 4(3): p. 263-272.
9. Kim, E., et al., Deficiency of a protein-repair enzyme results in the accumulation of altered proteins, retardation of growth, and fatal seizures in mice. Proceedings of the National Academy of Sciences of the United States of America., 1997. 94(12): p. 6132-7.
10. Shah, N. K., et al., Structural consequences of D-amino acids in collagen triple-helical peptides. Biopolymers, 1999. 49(4): p. 297-302.
11. Maroudas, A., G. Palla, and E. Gilav, Racemization of aspartic acid in human articular cartilage. Connect Tissue Res, 1992. 28(3): p. 161-9.
12. Brinton, K. L., et al., Aspartic acid racemization and age-depth relationships for organic carbon in Siberian permafrost. Astrobiology, 2002. 2(1): p. 77-82.
13. Ohtani, S., Different racemization ratios in dentin from different locations within a tooth. Growth Dev Aging, 1997. 61(2): p. 93-9.
14. Ohtani, S., S. Kato, and H. Sugeno, Changes in D-aspartic acid in human deciduous teeth with age from 1-20 years. Growth Dev Aging, 1996. 60(1): p. 1-6.
15. Helfman, P. M. and J. L. Bada, Aspartic acid racemization in tooth enamel from living humans. Proc Natl Acad Sci USA, 1975. 72(8): p. 2891-4.
16. Verzijl, N., et al., Effect of collagen turnover on the accumulation of advanced glycation end products. J Biol Chem, 2000. 275(50): p. 39027-31.
17. Maroudas, A., et al., Aggrecan turnover in human articular cartilage: use of aspartic acid racemization as a marker of molecular age. Arch Biochem Biophys, 1998. 350(1): p. 61-71.
22. Niyibizi, C., P. P. Fietzek, and M. van der Rest, Human placenta type V collagens. Evidence for the existence of an alpha 1(V) alpha 2(V) alpha 3(V) collagen molecule. Journal of Biological Chemistry., 1984. 259(22): p. 14170-4.
23. Ayad, S., et al., The Extracellular Matrix Facts Book. 1994, San Diego: Academic Press. 34-36.
24. Kraus, V., et al., Production and characterization of a novel monoclonal antibody to the G3 domain of cartilage aggrecan. Tran Orthop Res, 1996. 21(2): p. 763.
25. Bello, A. E., et al., Comparison of synovial fluid cartilage marker concentrations and chondral damage assessed arthroscopically in acute knee injury. Osteoarthritis & Cartilage, 1997. 5(6): p. 419-26.
26. Ayral, X., et al., Arthroscopic evaluation of chondropathy in osteoarthritis of the knee. J Rheum, 1996. 23(4): p. 698-706.
27. Kraus, V., N. Gell, and H. Wang, Pilot study of bone and cartilage serum biomarkers with aquatic exercise. Arthritis Research Conference (co-sponsored by AF, NIAMS, ACR), 1997: p. 56.
28. Lindhorst, E., et al., Longitudinal characterization of the canine meniscectomy model of osteoarthritis with cartilage biomarkers. Trans Orthop Res Soc, 1997. 22: p. 425.
29. Lindhorst, E., et al., Allograft reconstruction of the medial meniscus prevents synovial fluid biomarker elevations following total meniscectomy. Trans Ortho Res Soc, 1998. 23(1): p. 156.
30. Huebner, J. L., et al., Collagenase 1 and collagenase 3 expression in a guinea pig model of osteoarthritis. Arthritis & Rheumatism, 1998. 41(5): p. 877-90.
31. Huebner, J. L., et al., A comparative analysis of bone and cartilage metabolism in two strains of guinea-pig with varying degrees of naturally occurring osteoarthritis. Osteoarthritis & Cartilage, 2002. 10(10): p. 758-767.
32. Huebner, J. L., V. V., and V. B. Kraus, Synovial fluid biomarkers as predictors of therapeutic efficacy in a longitudinal analysis of the Hartley guinea pig model of naturally occurring osteoarthritis. Trans Orthop Res Soc, 2003. 28: p. 760.
33. Huebner, J. L., J. M. Williams, and V. B. Kraus, The time course of collagen fibril disruption in primary guinea pig osteoarthritis. Trans Orthop Res Soc, 2003. 28: p. 748.
34. Clark, A. G., et al., Serum cartilage oligomeric matrix protein reflects osteoarthritis presence and severity: the Johnston County Osteoarthritis Project. Arthritis Rheum, 1999. 42(11): p. 2356-64.
35. Dragomir, A. D., et al., Serum cartilage oligomeric matrix protein and clinical signs and symptoms of potential pre-radiographic hip and knee pathology. Osteoarthritis & Cartilage., 2002. 10(9): p. 687-91.
36. Jordan, J. M., et al., Ethnic and sex differences in serum levels of cartilage oligomeric matrix protein: the Johnston County Osteoarthritis Project. Arthritis & Rheumatism., 2003. 48(3): p. 675-81.
37. Vilim, V., et al., Serum cartilage oligomeric matrix protein reflects the presence of clinically diagnosed synovitis in patients with knee osteoarthritis. Osteoarthritis & Cartilage, 2001. 9(7): p. 612-8.
38. Vilim, V., et al., Serum levels of cartilage oligomeric matrix protein (COMP) correlate with radiographic progression of knee osteoarthritis. Osteoarthritis & Cartilage., 2002. 10(9): p. 707-13.
39. Vilim, V., et al., Monoclonal antibodies to human cartilage oligomeric matrix protein (COMP): epitope mapping and characterization of sandwich ELISA. Clinica Chimica Acta, 2003. 328: p. 59-69.
40. Dragomir, A., et al., Serum cartilage oligomeric matrix protein (COMP) levels and current hormone replacement therapy (HRT) in postmenopausal African-American and Caucasian women. Arthritis & Rheumatism, 2002. 46 (Suppl 9).
41. Carlson, C. S., et al., Synovial fluid biomarker levels predict articular cartilage damage following complete medial meniscectomy in the canine knee. Journal of Orthopaedic Research, 2002. 20(1): p. 92-100.
42. Kraus, V., et al., Urea as a passive transport marker for arthritis biomarker studies. Arthritis Rheum, 2002. 46(2): p. 420-427.
43. Jordan, J., et al., Serum hyaluronic acid (HA) levels and radiographic knee osteoarthritis (OA). Arthritis & Rheumatism, 2002. 46 (Suppl 9).
44. Jordan, J. M., et al., Serum C-Reactive Protein (CRP) levels and radiographic osteoarthritis (OA) in African-Americans and Caucasians. Arthritis & Rheumatism, 2002.46 (Suppl 9): p. S373.
45. Miller, E. J., Structural studies on cartilage collagen employing limited cleavage and solubilization with pepsin. Biochemistry, 1972. 11(26): p. 4903-9.
46. Eyre, D. and H. Muir, Characterisation of the major CNBr-derived peptides of porcine type II collagen. Connective Tissue Res, 1975. 3: p. 165-170.
47. Verzijl, N., et al., Age-related accumulation of the advanced glycation endproduct pentosidine in human articular cartilage aggrecan: the use of pentosidine levels as a quantitative measure of protein turnover. Matrix Biol, 2001. 20(7): p. 409-17.
48. Farndale, R., C. Sayers, and A. Barrett, A direct spectrophotometric microassay for sulphated glycosaminoglycans in cartilage cultures. Connective Tissue Res, 1982.9: p. 247-248.
49. Garnero, P., et al., Cross sectional evaluation of biochemical markers of bone, cartilage, and synovial tissue metabolism in patients with knee osteoarthritis: relations with disease activity and joint damage. Ann Rheum Dis, 2001. 60(6): p. 619-26.
50. Bornstein, P., Structurally distinct collagen types. Ann Rev Biochem, 1980. 49(1980): p. 957-1003.
51. Miller, E. and R. Rhodes, Preparation and characterization of the different types of collagen. Methods Enzymol, 1982. 82: p. 51-59.
52. Rowley, M., et al., Comparative epitope mapping of antibodies to collagen in women with silicone breast implants, systemic lupus erythematosis and rheumatoid arthritis. j Autoimmunity, 1994. 8: p. 777-787.
53. Jordan, J. M., et al., The impact of arthritis in rural populations. Arthritis Care and Res, 1995. 8(4): p. 242-250.
54. Jordan, J. M., et al., Ethnic differences in self-reported functional status in the rural south: the Johnston County Osteoarthritis Project. Arthritis Care & Research, 1996. 9(6): p. 483-91.
55. Jordan, J. M., et al., Self-reported functional status in osteoarthritis of the knee in a rural southern community: the role of sociodemographic factors, obesity, and knee pain. Arthritis Care Res, 1996. 9(4): p. 273-8.
56. Jordan, J., et al., Knee osteoarthritis is different in African-Americans than in Caucasians. Arthritis Rheum, 1997. 40: p. S236.
57. Jordan, J., et al., Knee pain and knee osteoarthritis severity in self-reported task specific disability: the Johnston County Osteoarthritis Project. Journal of Rheumatology, 1997. 24(7): p. 1344-9.
58. Scott, W. J., et al., Reliability of grading scales for individual radiographic features of osteoarthritis of the knee: the Baltimore Longitudinal Study of Aging atlas of knee osteoarthritis. Invest Radiol, 1993. 28: p. 497-50.
59. Heinegard and Paulsson Structure and metabolism of proteoglycans In Extracellular Matrix Biochemistry. Eds. Piez and Reddi. Elsevier, New York, 1984. pp. 277-328.
60. Rodriguez-Garcia et al. Increased concentrations of serum pentosidine in rheumatoid arthritis. Clin Chem, 1998. 44(2):250-5.
61. Hashimoto et al. Determination of free amino acid enantiomers in rat brain and serum by high-performance liquid chromatography after derivatization with N-tert.-butyloxycarbonyl-L-cysteine and o-phthaldialdehyde. J Chromatography, 1992. 582:41-48.
62. Kraus et al. Urinary type II collagen neoepitope as an outcome measure for relapsing polychondritis. Arthritis Rheum, 2003. 48(10):2942-2948.
63. Saudek and Kay. Advanced glycation end products and osteoarthritis. Current Rheumatology Reports, 2003. 5(1):33-40.

64. Onorato et al. Immunohistochemical and ELISA assays for biomarkers of oxidative stress in aging and disease. Annals of the New York Academy of Sciences, 1998. 854:277-90.

TABLE 1
Collagen 2 and D-Asp immunoreactivity in collagen fragments from
urine (immunoreactivity is expressed as the fold difference
from background calculated by the ratio of sample OD to
background OD; RP = relapsing polychondritis).

| Subject | Disease State | Sample | Dilution | Collagen 2 | D-Asp |
|---|---|---|---|---|---|
| Adult 73 yr | OA | urine | undiluted | 20.33 | 1.65 |
| old female | | | | | |
| (Caucasian) | | | | | |
| Adult 52 yr | OA | urine | undiluted | 20.28 | 2.04 |
| old male | | | | | |
| (Caucasian) | | | | | |
| 19 years old | Active RP | urine | undiluted | 11.28 | 10.06 |
| 19 years old | Remission | urine | undiluted | 3.77 | 1.24 |
| | RP | | | | |
| 10 years old | Healthy | urine | undiluted | 11.58 | 1.24 |

TABLE 2
D-Asp immunoreactivity in aggrecan fragments from serum
treated with chondroitinase ABC (immunoreactivity is
expressed as the fold difference from background calculated
by the ratio of sample OD to background OD).

| Subject | Disease State | Sample | Dilution | D-Asp |
|---|---|---|---|---|
| Pooled adult | unknown | serum | 1:1.4 | 3.68 |
| Adult 82 yr | OA | serum | 1:1.4 | 1.59 |
| old male | | | | |
| (African | | | | |
| American) | | | | |
| Adult 86 yr | OA | serum | 1:1.4 | 1.21 |
| old (African | | | | |
| American) | | | | |

1. A method of determining, in a sample, the proportion of a total amount of a molecule that is derived from catabolism due to the presence of age-related molecular alterations on the molecule, comprising:
a) determining the total amount of the molecule in the sample;
b) determining the amount of the molecule in the sample that contains D-aspartate; and
c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

2. The method of claim 1, wherein the molecule is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

3. The method of claim 1, wherein the sample is selected from the group consisting of synovial fluid, urine, serum, plasma, cells and tissue.

4. A method of determining, in a sample, the proportion of a total amount of a molecule that is derived from catabolism due to the presence of age-related molecular alterations on the molecule, comprising:
a) determining the total amount of the molecule in the sample;
b) determining the amount of the molecule in the sample that contain an advanced glycation end product; and
c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

5. The method of claim 4, wherein the molecule is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

6. The method of claim 4, wherein the sample is selected from the group consisting of synovial fluid, urine, serum, plasma, cells and tissue.

7. The method of claim 4, wherein the advanced glycation end product is selected from the group consisting of: pentosidine, N(epsilon)-(carboxymethyl)lysine, N(epsilon)-(carboxyethyl)lysine, imidazolone, and pyrraline.

8. A method of determining, in a sample, the proportion of a total amount of a molecule that is derived from catabolism due to the presence of age-related molecular alterations on the molecule, comprising:
a) determining the total amount of the molecule in the sample;
b) determining the amount of the molecule in the sample that contains D-aspartate and the amount of the molecule in the sample that contains an advanced glycation end product; and
c) calculating the proportion of the amount of the molecule of step (b) relative to the total amount of the molecule as determined in step (a), thereby determining the proportion of the total amount of the molecule that is derived from catabolism due to the presence of age-related molecular alterations in the molecule.

9. The method of claim 8, wherein the molecule is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

10. The method of claim 8, wherein the sample is selected from the group consisting of synovial fluid, urine, serum, plasma, cells and tissue.

11. The method of claim 8, wherein the advanced glycation end product is selected from the group consisting of: pentosidine, N(epsilon)-(carboxymethyl)lysine, N(epsilon)-(carboxyethyl)lysine, imidazolone, and pyrraline.

12. A method of diagnosing a musculoskeletal, arthritic or joint disorder in a subject, comprising:
a) measuring an amount of D-aspartate in a sample of the subject; and
b) comparing the amount of D-aspartate in the sample of (a) with an amount of D-aspartate in a sample of a control subject, whereby an increased amount of D-aspartate in the sample of the subject as compared to the amount of D-aspartate in the sample of the control subject is diagnostic of a musculoskeletal, arthritic or joint disorder in the subject.

13. The method of claim 12, wherein the sample is a body fluid selected from the group consisting of synovial fluid, urine, serum and plasma.

14. The method of claim 12, wherein the sample is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

15. The method of claim 12, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

16. A method of diagnosing a musculoskeletal, arthritic or joint disorder in a subject, comprising:
a) measuring an amount of an advanced glycation end product in a sample of the subject; and
b) comparing the amount of the advanced glycation end product in the sample of (a) with an amount of an advanced glycation end product in a sample of a control subject, whereby an increased amount of an advanced glycation end product in the sample of the subject as compared to the amount of an advanced glycation end product in the sample of the control subject is diagnostic of a musculoskeletal, arthritic or joint disorder in the subject.

17. The method of claim 16, wherein the sample is a body fluid selected from the group consisting of synovial fluid, urine, serum and plasma.

18. The method of claim 16, wherein the sample is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

19. The method of claim 16, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

20. The method of claim 16, wherein the advanced glycation end product is selected from the group consisting of: pentosidine, N(epsilon)-(carboxymethyl)lysine, N(epsilon)-(carboxyethyl)lysine, imidazolone, and pyrraline.

21. A method of diagnosing a musculoskeletal, arthritic or joint disorder in a subject, comprising:
a) measuring an amount of D-aspartate and an amount of an advanced glycation end product in a sample of the subject; and
b) comparing the amount of the D-aspartate and the amount of the advanced glycation end product in the sample of (a) with an amount of D-aspartate and an amount of an advanced glycation end product in a sample of a control subject, whereby an increased amount of D-aspartate and of an advanced glycation end product in the sample of the subject as compared to the amount D-aspartate and of an advanced glycation end product in the sample of the control subject is diagnostic of a musculoskeletal, arthritic or joint disorder in the subject.

22. The method of claim 21, wherein the sample is a body fluid selected from the group consisting of synovial fluid, urine, serum and plasma.

23. The method of claim 21, wherein the sample is a joint tissue molecule selected from the group consisting of cartilage oligomeric matrix protein (COMP), link protein, t type I, II, III, V, VI, IX, X, XI and XII collagens, aggrecan, glycosaminoglycan, link protein and any combination thereof.

24. The method of claim 21, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

25. The method of claim 21, wherein the advanced glycation end product is selected from the group consisting of: pentosidine, N(epsilon)-(carboxymethyl)lysine, N(epsilon)-(carboxyethyl)lysine, imidazolone, and pyrraline.

26. A method of diagnosing a musculoskeletal, arthritic or joint disorder in a subject, comprising:
a) determining the proportion of a total amount of a molecule in the subject that is derived from a catabolic process comprising:
i) determining the total amount of the molecule in a joint tissue sample from the subject;
ii) determining the amount of the molecule in the sample that contains D-aspartate; and
iii) calculating the proportion of the amount of the molecule of step (ii) relative to the total amount of the molecule as determined in step (i), thereby determining the proportion derived from a catabolic process; and
b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic or joint disorder in the subject.

27. The method of claim 26, wherein the joint tissue molecule is selected from the group consisting of cartilage oligomeric matrix protein (COMP), link protein, type I, II, III, V, VI, IX, X, XI and XII collagens, aggrecan, glycosaminoglycan, link protein and any combination thereof.

28. The method of claim 26, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

29. A method of diagnosing a musculoskeletal, arthritic or joint disorder in a subject, comprising:
a) determining the proportion of a total amount of a molecule in the subject that is derived from a catabolic process comprising:
i) determining the total amount of the molecule in a joint tissue sample from the subject;
ii) determining the amount of the molecule in the sample that contains an advanced glycation end product; and
iii) calculating the proportion of the amount of the molecule of step (ii) relative to the total amount of the molecule as determined in step (i), thereby determining the proportion derived from a catabolic process; and
b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic or joint disorder in the subject.

30. The method of claim 29, wherein the joint tissue molecule is selected from the group consisting of cartilage oligomeric matrix protein (COMP), link protein, type I, II, III, V, VI, IX, X, XI and XII collagens, aggrecan, glycosaminoglycan, link protein and any combination thereof.

31. The method of claim 29, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

32. The method of claim 29, wherein the advanced glycation end product is selected from the group consisting of: pentosidine, N(epsilon)-(carboxymethyl)lysine, N(epsilon)-(carboxyethyl)lysine, imidazolone, and pyrraline.

33. A method of diagnosing a musculoskeletal, arthritic or joint disorder in a subject, comprising:
a) determining the proportion of a total amount of a molecule in the subject that is derived from a catabolic process comprising:
i) determining the total amount of the molecule in a joint tissue sample from the subject;
ii) determining the amount of the molecule in the sample that contains D-aspartate and the amount of the molecule in the sample that contains an advanced glycation end product; and
iii) calculating the proportion of the amount of the molecule of step (ii) relative to the total amount of the molecule as determined in step (i), thereby determining the proportion derived from a catabolic process; and
b) comparing the proportion of the molecule in the subject that is derived from a catabolic process with the proportion of the molecule in a control subject that is derived from a catabolic process, whereby an increased proportion in the subject as compared to the proportion in the control subject is diagnostic of a musculoskeletal, arthritic or joint disorder in the subject.

34. The method of claim 33, wherein the joint tissue molecule is selected from the group consisting of cartilage oligomeric matrix protein (COMP), link protein, type I, II, III, V, VI, IX, X, XI and XII collagens, aggrecan, glycosaminoglycan, link protein and any combination thereof.

35. The method of claim 33, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

36. The method of claim 33, wherein the advanced glycation end product is selected from the group consisting of: pentosidine, N(epsilon)-(carboxymethyl)lysine, N(epsilon)-(carboxyethyl)lysine, imidazolone, and pyrraline.

37. A method of identifying a subject at risk of developing a musculoskeletal, arthritic or joint disorder, comprising:
a) measuring an amount of D-aspartate in a sample of the subject; and
b) comparing the amount of D-aspartate in the sample of (a) with an amount of D-aspartate in a sample of a control subject, whereby an increased amount of D-aspartate in the sample of the subject as compared to the amount of D-aspartate in the sample of the control subject identifies a subject at risk of developing a musculoskeletal, arthritic or joint disorder.

38. The method of claim 37, wherein the sample is a body fluid selected from the group consisting of synovial fluid, urine, serum and plasma.

39. The method of claim 37, wherein the sample is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

40. The method of claim 37, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

41. A method of identifying a subject at risk of developing a musculoskeletal, arthritic or joint disorder, comprising:
a) measuring an amount of an advanced glycation end product in a sample of the subject; and
b) comparing the amount of the advanced glycation end product in the sample of (a) with an amount of an advanced glycation end product in a sample of a control subject, whereby an increased amount of an advanced glycation end product in the sample of the subject as compared to the amount of an advanced glycation end product in the sample of the control subject identifies a subject at risk of developing a musculoskeletal, arthritic or joint disorder.

42. The method of claim 41, wherein the sample is a body fluid selected from the group consisting of synovial fluid, urine, serum and plasma.

43. The method of claim 41, wherein the sample is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

44. The method of claim 41, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

45. The method of claim 41, wherein the advanced glycation end product is selected from the group consisting of: pentosidine, N(epsilon)-(carboxymethyl)lysine, N(epsilon)-(carboxyethyl)lysine, imidazolone, and pyrraline.

46. A method of identifying a subject at risk of developing a musculoskeletal, arthritic or joint disorder, comprising:
a) measuring an amount of D-aspartate and an amount of an advanced glycation end product in a sample of the subject; and
b) comparing the amount of the D-aspartate and the amount of the advanced glycation end product in the sample of (a) with an amount of D-aspartate and an amount of an advanced glycation end product in a sample of a control subject, whereby an increased amount of D-aspartate and of an advanced glycation end product in the sample of the subject as compared to the amount D-aspartate and of an advanced glycation end product in the sample of the control subject identifies a subject at risk of developing a musculoskeletal, arthritic or joint disorder.

47. The method of claim 46, wherein the sample is a body fluid selected from the group consisting of synovial fluid, urine, serum and plasma.

48. The method of claim 46, wherein the sample is a joint tissue molecule selected from the group consisting of cartilage oligomeric matrix protein (COMP), link protein, type I, II, III, V, VI, IX, X, XI and XII collagens, aggrecan, glycosaminoglycan, link protein and any combination thereof.

49. The method of claim 46, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

50. The method of claim 46, wherein the advanced glycation end product is selected from the group consisting of: pentosidine, N(epsilon)-(carboxymethyl)lysine, N(epsilon)-(carboxyethyl)lysine, imidazolone, and pyrraline.

51. A method of identifying a subject at risk of developing a musculoskeletal, arthritic or joint disorder, comprising:
a) determining the proportion of a total amount of a molecule in the subject that is derived from a catabolic process comprising:
i) determining the total amount of the molecule in a joint tissue sample from the subject;
ii) determining the amount of the molecule in the sample that contains D-aspartate; and
iii) calculating the proportion of the amount of the molecule of step (ii) relative to the total amount of the molecule as determined in step (i), thereby determining the proportion derived from a catabolic process; and
b) comparing the proportion of the molecule in the subject that is derived from a catabolic process with the proportion of the molecule in a control subject that is derived from a catabolic process, whereby an increased proportion in the subject as compared to the proportion in the control subject identifies a subject at increased risk of developing a musculoskeletal, arthritic or joint disorder.

52. The method of claim 51, wherein the joint tissue molecule is selected from the group consisting of cartilage oligomeric matrix protein (COMP), link protein, type I, II, III, V, VI, IX, X, XI and XII collagens, aggrecan, glycosaminoglycan, link protein and any combination thereof.

53. The method of claim 51, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

54. A method of identifying a subject at risk of developing a musculoskeletal, arthritic or joint disorder, comprising:
a) determining the proportion of a total amount of a molecule in the subject that is derived from a catabolic process comprising:
i) determining the total amount of the molecule in a joint tissue sample from the subject;
ii) determining the amount of the molecule in the sample that contains an advanced glycation end product; and
iii) calculating the proportion of the amount of the molecule of step (ii) relative to the total amount of the molecule as determined in step (i), thereby determining the proportion derived from a catabolic process; and
b) comparing the proportion of the molecule in the subject that is derived from a catabolic process with the proportion of the molecule in a control subject that is derived from a catabolic process, whereby an increased proportion in the subject as compared to the proportion in the control subject identifies a subject at risk of developing a musculoskeletal, arthritic or joint disorder.

55. The method of claim 54, wherein the joint tissue molecule is selected from the group consisting of cartilage oligomeric matrix protein (COMP), link protein, type I, II, III, V, VI, IX, X, XI and XII collagens, aggrecan, glycosaminoglycan, link protein and any combination thereof.

56. The method of claim 54, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

57. The method of claim 54, wherein the advanced glycation end product is selected from the group consisting of: pentosidine, N(epsilon)-(carboxymethyl)lysine, N(epsilon)-(carboxyethyl)lysine, imidazolone, and pyrraline.

58. A method of identifying a subject at risk of developing a musculoskeletal, arthritic or joint disorder, comprising:
a) determining the proportion of a total amount of a molecule in the subject that is derived from a catabolic process comprising:
i) determining the total amount of the molecule in a joint tissue sample from the subject;
ii) determining the amount of the molecule in the sample that contains D-aspartate and the amount of the molecule in the sample that contains an advanced glycation end product; and
iii) calculating the proportion of the amount of the molecule of step (ii) relative to the total amount of the molecule as determined in step (i), thereby determining the proportion derived from a catabolic process; and
b) comparing the proportion of the molecule in the subject that is derived from a catabolic process with the proportion of the molecule in a control subject that is derived from a catabolic process, whereby an increased proportion in the subject as compared to the proportion in the control subject is identified as a subject at risk of developing a musculoskeletal, arthritic or joint disorder.

59. The method of claim 58, wherein the joint tissue molecule is selected from the group consisting of cartilage oligomeric matrix protein (COMP), link protein, type I, II, III, V, VI, IX, X, XI and XII collagens, aggrecan, glycosaminoglycan, link protein and any combination thereof.

60. The method of claim 58, wherein the musculoskeletal, arthritic or joint disorder is selected from the group consisting of osteoarthritis, rheumatoid arthritis, psoriatic arthritis, ankylosing spondylitis, gout, crystalline arthritis, arthritis of unknown etiology, joint injury and relapsing polychondritis.

61. The method of claim 58, wherein the advanced glycation end product is selected from the group consisting of: pentosidine, N(epsilon)-(carboxymethyl)lysine, N(epsilon)-(carboxyethyl)lysine, imidazolone, and pyrraline.
